(19) United States
(12) Patent Application Publication
(10) Pub. No.: US 2025/0169907 A1
(43) Pub. Date: May 29, 2025

(54) CONTINUUM ROBOT, HUB ASSEMBLY, AND METHOD OF MANUFACTURE

(71) Applicant: CANON U.S.A., INC., MELVILLE, NY (US)

(72) Inventors: Inderpal Singh, Norwood, MA (US); Anne Yujin Edge, Somerville, MA (US)

(21) Appl. No.: 18/947,673

(22) Filed: Nov. 14, 2024

Related U.S. Application Data
(60) Provisional application No. 63/603,419, filed on Nov. 28, 2023.
(60) Provisional application No. 63/603,561, filed on Nov. 28, 2023.
(60) Provisional application No. 63/603,578, filed on Nov. 28, 2023.

(51) Int. Cl. A61B 34/00; A61B 34/30; A61M 25/01

(52) U.S. Cl. CPC A61B 34/71; A61B 2034/301; A61M 25/0113; A61M 25/0147

(57) ABSTRACT

Disclosed are a robotic apparatus, a catheter and a continuum robot hub for use with same, and a methods for use and manufacture. The continuum robot hub includes a distal end, a proximal end, a section extending from the distal end to the proximal end, and a plurality of hub guide channels that each extend across a distal pitch diameter change and a proximal pitch diameter change along an exterior surface of the section. The distal pitch diameter change extends from the distal end along a first length of the hub, and the proximal pitch diameter change extends from a proximal end of the distal pitch diameter change along a second length of the hub.

### CROSS-REFERENCE TO RELATED APPLICATION(S)

This application claims the priority under 35 U.S.C. § 119(e) to U.S. Provisional Application No. 63/603,419 (Docket 2600-30862-prov), to U.S. Provisional Application No. 63/603,561 (Docket 2600-30660-prov), and to U.S. Provisional Application No. 63/603,578 (Docket 2600-30912-prov), each of which were filed on Nov. 28, 2023, the entire disclosure of each of which is incorporated herein by reference.

### FIELD OF THE DISCLOSURE

The present disclosure relates generally to continuum robots applicable to guide devices, including medical devices, interventional tools, instruments, and endoscopes.

### BACKGROUND OF THE DISCLOSURE

A continuum robot (also referred to as a snake) includes a plurality of bending sections having a flexible structure, with the shape of the continuum robot being controlled by deforming the bending sections. The snake has significant advantages over existing robots including rigid link robots. An advantage is that the snake can move along a curve in a narrow space or in an environment with scattered objects in which the rigid link robot may get stuck. Another advantage is that it is possible to operate the snake without damaging surrounding fragile elements, utilizing intrinsic flexibility of the snake.

In recent years, minimally invasive medical care, with which burden on the patient can be reduced and quality of life after treatment or inspection can be improved, has been attracting attention. A surgery or inspection using an endoscope is a typical example of minimally invasive medical care. For example, a laparoscopic surgery is advantageous over a conventional abdominal surgery in that it can be performed with a smaller surgical wound, which results in a shorter stay in the hospital and less damage to the appearance.

Endoscopes used for the minimally invasive medical care are roughly divided into rigid endoscopes and soft endoscopes. Although a rigid endoscope may provide clear images, the direction in which an observation target can be observed is limited. In addition, when the rigid endoscope is inserted into a curved organ, e.g., esophagus, large intestine, or urethra, an insertion portion of the rigid endoscope may press on the organ. In contrast, a soft endoscope includes an insertion portion formed of a bendable member, so that a large area can be observed in detail by adjusting the bending angle of the distal end of the endoscope. In addition, by bending the insertion portion along an insertion path, burden on the patient can be reduced. When the number of bendable portions is increased, the endoscope can be inserted to a deep area of the body without causing the endoscope to come into contact with tissue even when the insertion path has a complex curved shape.

Accordingly, soft endoscopes having a plurality of bendable portions have been researched and developed.

Various related art disclosures in the field include U.S. Pat. No. 11,559,190, which discusses a steerable device with push-pull actuators and breakout unit, as well as WO 2022/146751 which discusses a steerable snake with push-pull rod structure. U.S. 2022/0202277 discusses a medical apparatus having a bendable body with a driving wire, a/k/a tendon; a break-out wire attached to the driving wire, with a distal end of the break-out wire attached to a proximal end of the driving wire; a distal guide tube guiding the driving wire and ending before the break-out wire with a space; a resilient element abutting the driving wire along at least a portion of a longitudinal direction of the driving wire; and an actuator configured to retract and advance the driving wire via the break-out wire thereby maneuvering the bendable body. Each of the afore-mentioned disclosures are incorporated herein by reference.

When controlling the bendable medical device by pushing or pulling the small diameter drive wires, the amount of operating force that can be applied to the drive wires is limited by a buckling force of the specific wire diameter and material. For the bendable medical device, space constraints imposed by target anatomy, tool dimensions, and the like, required use of small diameter wires. To prevent wire buckling, continuous support may be provided around the drive wires throughout the entire length of the bendable medical device. U.S. 2015/0142013 discusses releasing tension from the continuum robot pull wires with a button/command for the continuum robot shape to conform to the anatomy. U.S. 2019/0105468 also discusses problematic buckling, especially as the size/diameter of the snake robot is decreased. When controlling the bendable medical device by pushing/pulling small diameter drive wires, the amount of operating force that can be applied to the drive wires is limited by wire buckling. Use of small diameter wires is important in bendable medical devices due to space constraints from the target anatomy, tool dimensions, etc.

### SUMMARY

Thus, to prevent wire buckling, continuous support around the drive wires throughout an entire length of the bendable medical device is provided.

An aspect of the present disclosure provides a hub for a continuum robot, with the hub including a distal end, a proximal end, a section extending from the distal end to the proximal end, and a plurality of guide channels. Each guide channel of the plurality of guide channels extends along a surface of the section. The surface includes a distal pitch diameter change and a proximal pitch diameter change. The distal pitch diameter change extends from the distal end along a first length of the hub. The proximal pitch diameter change extends from a proximal end of the distal pitch diameter change along a second length of the hub.

Another aspect of the present disclosure provides a hub for connecting a continuum robot to a controller, with the hub including a distal end, a proximal end, and a section between the proximal end and the distal end. The distal end includes a first face with a first maximum length. The proximal end includes a second face with a second maximum length larger than the first maximum length.

A further aspect of the present disclosure provides an access component for a continuum robot, with the access component including a hub configured to fixedly attach to a shaft of the continuum robot, with the hub being configured to attach the continuum robot to a controller; and a hub cover, with a distal end of the hub including a first face with a diameter having a first maximum length, a proximal end of the hub including a second face with a diameter having a second maximum length, larger than the first maximum length, and hub hypotubes extending from the proximal end to the distal end, across the hub.

A still further aspect of the present disclosure provides a method for manufacturing a continuum robot that includes forming a hub body by aligning a hub cone with a tool channel extending through the hub body and a plurality of hub hypotubes extending along an exterior of the hub body; extending first ends of mandrels of a plurality of mandrels into respective hollows of hub hypotubes of the plurality of hub hypotubes; extending second ends of the mandrels into hollows of a respective support sleeve lumen of a plurality of support sleeve lumens that align with the respective hollows of the hub hypotube; and thermally bonding the hub body to the plurality of support sleeve lumens, with the thermal bonding of the hub body to the plurality of support sleeve lumens bonding the tool channel and the plurality of hub hypotubes to the hub body; and forming a channel in each aligned support sleeve lumen and hub hypotube by withdrawing the each mandrel of the plurality of mandrels from the respective hollows.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present innovation will become apparent from the following detailed description when taken in conjunction with the accompanying figures showing illustrative embodiments of the present innovation.

FIG. 1 is a block diagram of an exemplary medial system including ancillary components and a bendable medical device.

FIG. 2 illustrates components of a continuum robot.

FIG. 3 illustrates relative arrangement of the actuator and catheter shaft of the continuum robot.

FIG. 4A is a cut away view illustrating drive wire spacing within a hub body.

FIG. 4B is a cut away view illustrating drive wire spacing within a catheter shaft.

FIG. 5A is a perspective view of hub assembly.

FIG. 5B is a top view of the hub assembly.

FIG. 6 is a rear perspective view of the hub body illustrating the single part hub body having multiple channels.

FIG. 7 illustrates hub hypotube buckling.

FIG. 8 is a profile view illustrating components connecting to the catheter hub, according to the present disclosure.

FIG. 9 is a perspective view of the components connecting to the catheter hub, according to the present disclosure.

FIG. 10 is a perspective view of the proximal components connecting to the catheter hub, affixed in respective clamps of a controller, according to the present disclosure.

FIG. 11 is a cut away view of hub guide hypotubes and guide disks of the hub guide, according to the present disclosure.

FIG. 12A illustrates an extended cone cover assembled in a pre-reflowed condition, according to the present disclosure.

FIG. 12B illustrates the cone cover assembled on the hub body in a reflowed condition, according to the present disclosure.

FIG. 13A is a cutaway profile view of the hub body, according to the present disclosure.

FIG. 13B is a view of the hub cone, according to the present disclosure.

FIG. 14 is a perspective view of the hub cone with a cone cover disassembled therefrom, according to the present disclosure.

FIG. 15 is a cutaway profile view of the hub cone, cone cover and outer shell, according to the present disclosure.

FIG. 16 is an expanded view of FIG. 15, according to the present disclosure.

FIG. 17 is a side view of a hypotubes, a cone cover, a hub body and catheter shaft, according to the present disclosure.

FIG. 18 shows changes in pitch diameter across the hub cone, according to the present disclosure.

FIG. 19 is a cut away view illustrating hub hypotube positioning within a catheter shaft, according to the present disclosure.

FIG. 20 is a perspective view illustrating hub hypotube positioning within a catheter shaft, according to the present disclosure.

FIG. 21 is a cut away view illustrating hub hypotube positioning within a catheter shaft, according to another embodiment.

FIG. 22 is a cut away view of a connection between the catheter and the hub, according to the present disclosure.

FIG. 23 is a cut away view of a connection between the catheter and the hub, according to another embodiment.

FIG. 24 is a perspective view of a hub hypotube sleeve connection with the shaft, according to the present disclosure.

FIG. 25 is a perspective partial cut away view of a transition from a distal end of the hub cone to a proximal end of the catheter shaft, according to the present disclosure.

FIG. 26A is a cut away view at a transition between the support sleeve lumens and the drive wire lumens, according to the present disclosure.

FIG. 26B is a reverse view cut away view from FIG. 26a, according to the present disclosure.

FIG. 27 is perspective partial cut away view of a transition from a distal end of the hub cone to a proximal end of the catheter shaft, according to another embodiment.

FIG. 28 is a view of components extending from the outer shell to a proximal end of the catheter shaft 5, according to the present disclosure.

FIG. 29 is a detailed view of the tapered distal end of the proximal stiffener, accordingly to an embodiment.

FIGS. 30 and 31 are perspective partial cut away views of a transition from a distal end of the hub cone to a proximal end of the catheter shaft, according to another embodiment.

FIG. 32 is a perspective view of a reflowed proximal stiffener, accordingly to an embodiment.

FIG. 33 is a cut away view of components adjacent to the support sleeve lumens, before reflow, according to the present disclosure.

FIG. 34 is a cut away view of components adjacent to the support sleeve lumens, after reflow, according to the present disclosure.

FIGS. 35 and 36 are perspective partial cut away views of a transition from a distal end of the hub cone to a proximal end of the catheter shaft, according to another embodiment.

FIG. 37 shows a proximal flared stiffener positioned on a proximal side of the proximal stiffener, according to the present disclosure.

FIGS. 38A and 38B show assembly of the inner cone, according to the present disclosure.

FIGS. 39A and 39B show the proximal flared stiffener and the proximal stiffener, according to the present disclosure.

FIGS. 40 and 41 show the tool channel extending from the hub cone through the catheter shaft, according to the present disclosure.

FIG. 42 is a perspective view of the cone cover on the hub cone with the flared proximal end and the proximal stiffener, according to the present disclosure.

FIG. 43 is a side view of the cone cover on the hub cone with the flared proximal end and the proximal stiffener, according to the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION

The resent disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

In the subject disclosure, systems and mechanisms of a continuum robot are described, followed by continuum robot support elements for reducing buckling, as well as the systems and procedures associated with the continuum robot and said support elements.

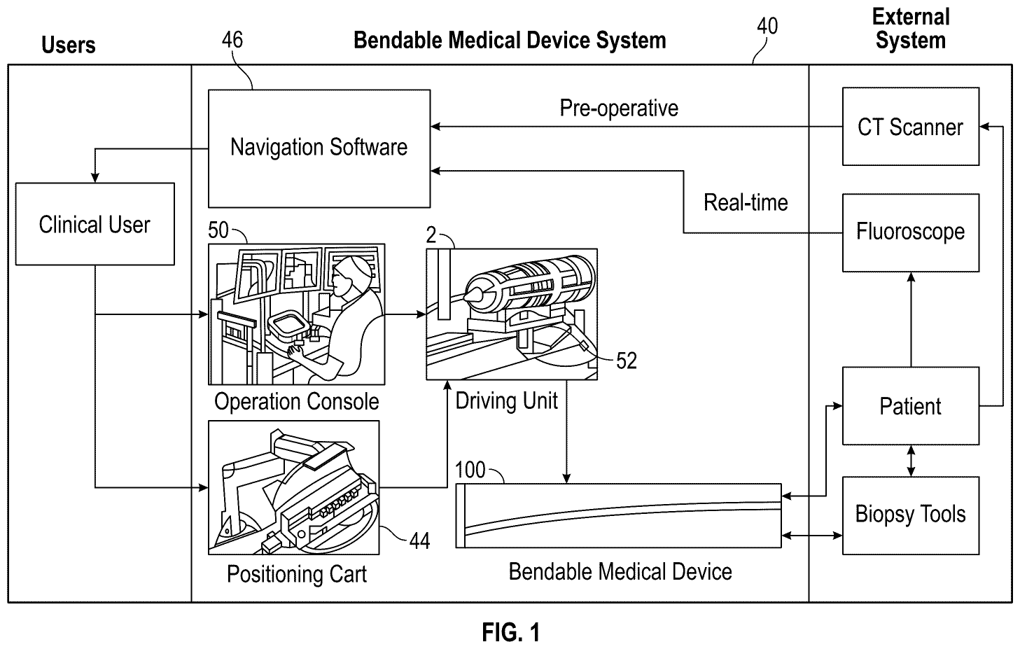

FIG. 1 is a block diagram of an exemplary medial system including ancillary components and a bendable medical device.

As illustrated in FIG. 1, the system 40 includes a driving unit 2 (also referred to as an actuator or driver) for driving the drive wires or tendons (also referred to as drive wire, drive wires, or driver(s)), and having a base stage 52, a continuum robot 100 (also referred to as bendable medical device, steerable catheter, snake, or robotic catheter), a positioning cart 44, an operation console 50 (also referred to as controller or control system), having push-button, thumb-stick, and/or joystick, and navigation software 46. The medical device system 40 is capable of interacting with external system components and clinical users to facilitate use in a patient.

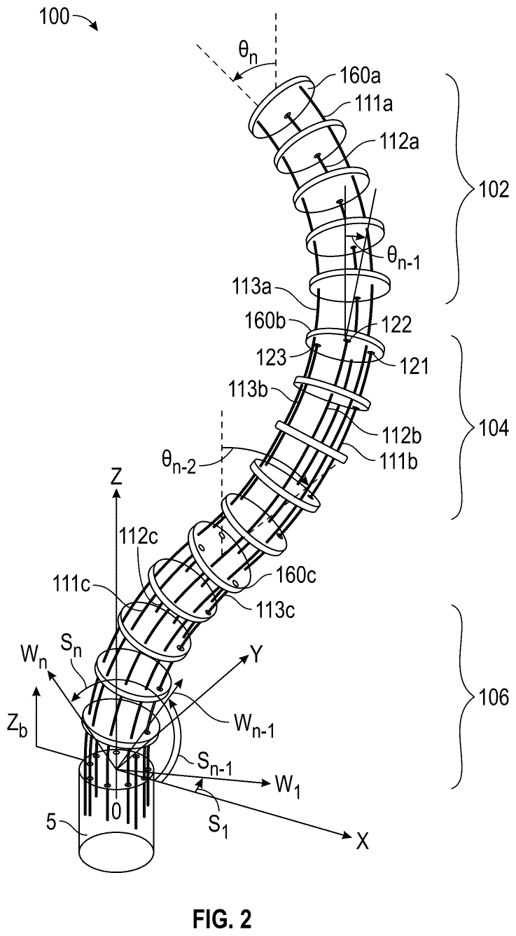

FIG. 2 illustrates components of a continuum robot.

As shown in FIG. 2, the continuum robot 100 includes push/pull drive wires 111b, 112b and 113b, which are connected to connection portions 121, 122 and 123, respectively, found on an end disc 160b, for controlling the middle bending section 104. Additional drive wires (three for each of the other bendable sections 102 and 106) 111a, 111c, 112a, 112c, 113a, 113c, are attached at the distal ends of each bendable section 102 and 106, to the respective end disc 160a and 160c. Each bending section is operated similarly. Thus, the description of one bending section, i.e., the middle bending section 104, will be recognized to apply to the other sections. Posture of the bending section 104 is controlled by pushing and pulling the wires 111b to 113b by using actuators, with l_{d}=the length of the central axis a bending section; θ_{d}=the bending angle of the distal end; ζₙ=the rotational angle of the distal end; ρₙ=the radius of curvature of a bending section.

The continuum robot 100 attaches to a catheter shaft 5, which may be disposed on a base stage 52 (FIG. 1) and can be moved by the base stage 52 in the longitudinal direction, to advance, retard and/or retract the continuum robot 100 into a target structure by advancing, retarding and/or retracting the base stage 52.

An operational console 50 (FIG. 1) may indicate a driving amount to the base stage 52 and, independently, to the actuator 2. The operational console 50 may include dedicated hardware including a field-programmable gate array (FPGA) and the like; and/or may be a computer including a storage unit, a work memory, and a central processing unit (CPU). Where the operational console 50 is a computer, the storage unit may be a memory that stores a software program corresponding to a control system algorithm and the CPU may expand the program in the work memory, and may execute the program line by line, for the computer to function as the operational console 50. In either case, the operational console 50 may communicably connect with the base stage 52 and the actuator 2, and the operational console 50 may send signals representing the driving amount and configuration to these control targets, which may be imputed by an end user through push buttons, joystick or the like. Thus, the continuum robot 100 includes at least one distal bending section 102, with robotic control for insertion and removal of the continuum robot 100 from the target for operation during lung biopsies, medical procedures, and similar operations.

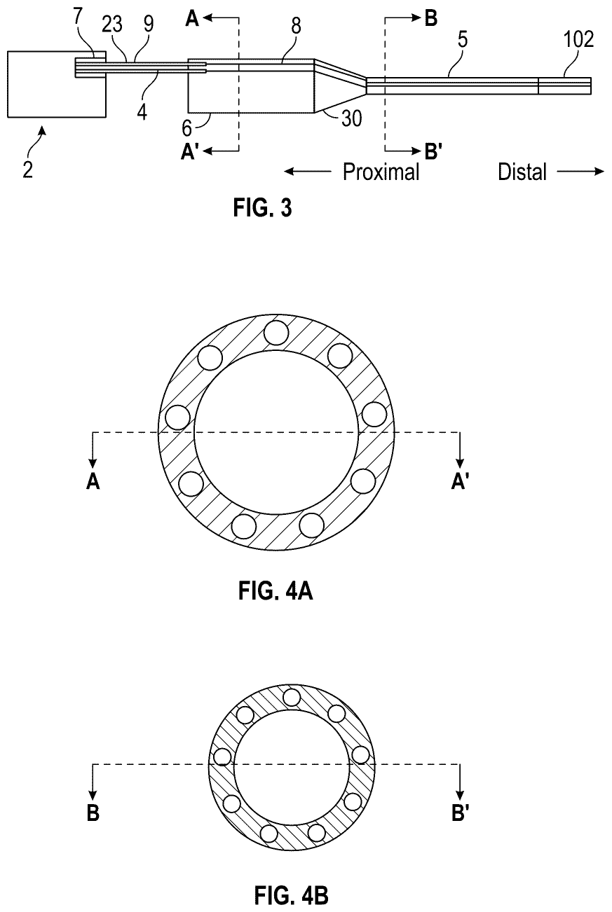

FIG. 3 illustrates relative arrangement of the actuator and catheter shaft of the continuum robot.

Posture and/or pose of the catheter shaft 5 may be controlled by push/pull on at least one drive wire 4. The catheter shaft 5 has at least one distal bending section 102, with at least three drive wires 4 terminating in each of the at least one distal bending section 102 to control bend angle and plane. The actuator 2 may selectively push/pull drive wires 4 to control the distal bending section 102. Pusher rods 9 on the catheter may be attached/detached by clamping performed by an actuator clamp 7 on the actuator 2, for removable attachment of a hub body 6 to the actuator 2 and controller (operation console 50). The pusher rods 9 attach/detach from respective clamps 7, with the pusher rods 9 being fixedly attached to the proximal end of respective driving wires 4. The drive wires 4 may slide within respective hub hypotubes 8, also referred to as support sleeves, which are anchored at distal ends of the hub body 6, and slide into the catheter shaft 5.

The hub body 6 may have a straight section at a proximal end thereof. The hub body 6 transitions from a proximal end of the pitch diameter that substantially aligns with the actuator clamps 7 (A-A′) to a distal end of the pitch diameter that substantially aligns with a diameter of the catheter shaft 5 (B-B′). The changes between such diameters may be referred to as pitch diameter transition 400.

At a proximal end of the transition, the hub hypotubes 8 connect to a push/pull assembly of the controller. For connection to the push pull assembly, supporting small wires and hypotubes which are pushed with up to 20 N force must be supported. Also, an increased wire diameter is used to removable clamping with high clamp strength via a pusher rod 9 that is to be clamped. For manufacturability, a total number of parts is to be minimized.

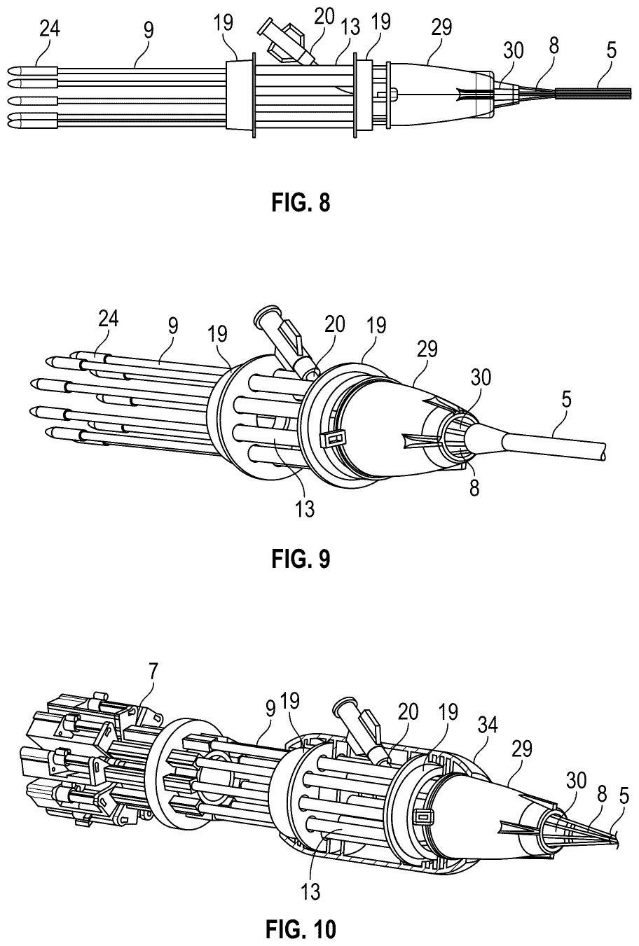

At the distal end, the hub hypotubes 8 connect to the catheter shaft and a strong bond between the hub and catheter shaft is created in limited space. The present disclosure also provides smooth and fully protected transition of the drive wires from the hub hypotubes 8 to lumens in the shaft 5. A sealed connection for a tool channel 20 (FIGS. 8 to 10) is created. Also provided is a gradual transition in stiffness from the larger rigid hub body to the smaller flexible catheter shaft.

FIG. 4A is a cut away view illustrating drive wire spacing.

FIG. 4A illustrates spacing between drive wires 4 within the hub body 6 that connects a controller to the shaft 5 of the continuum robot. The drive wires substantially following the pitch diameter. FIG. 4A illustrates a proximal pitch diameter located at cutaway A-A′ (FIG. 3). A proximal end of the proximal pitch diameter aligns the drive wires 4 with the controller and respective actuation clamps 7 (FIG. 10) of the controller.

FIG. 4B is a cut away view drive wire spacing within a catheter shaft.

FIG. 4B illustrates spacing of drive wires 4 within the shaft 5 of the continuum robot at cutaway B-B′ (FIG. 3) that is aligned with the drive wires 4 within the catheter shaft 5. FIG. 4B illustrates a distal pitch diameter. A distal end of the distal pitch diameter aligns the drive wires 4 with respective lumens that traverse the hub body 6.

Comparison of FIG. 4A to FIG. 4B illustrates the change in diameters that the drive wires across the hub body 6. The transition between pitch diameters may limit push-ability due to pusher rod buckling in response to a pushing force applied by the controller and/or applied a distal end of the catheter 5. For example, if unsupported for more than 50 mm, a 26TW hypotube may buckle with less than 5 N. The drive wire 4 is no longer able to be accurately pushed once the hypotube 8 (FIGS. 8-12) is bent. The hypotube 8 cannot be restored to a perfectly straight condition, as needed for proper operation. The present disclosure provides an assembly that includes a cone cover 29 that prevents buckling of the hub hypotube 8 along the curved surface of the transition between diameters along the hub body 6. The cone cover 29 may be elastomeric and configured to stretch over the hub cone 6 and press hub hypotubes 8 into grooves, i.e., guide channels, and apply a radial force that prevent buckling of the hub hypotubes 8. The hub cone cover 29 may be bonded to the hub cone 6. The cone cover 29, hub cone 6, tool channel 20, catheter shaft 5, and hub hypotubes (8) may also be thermally bonded together

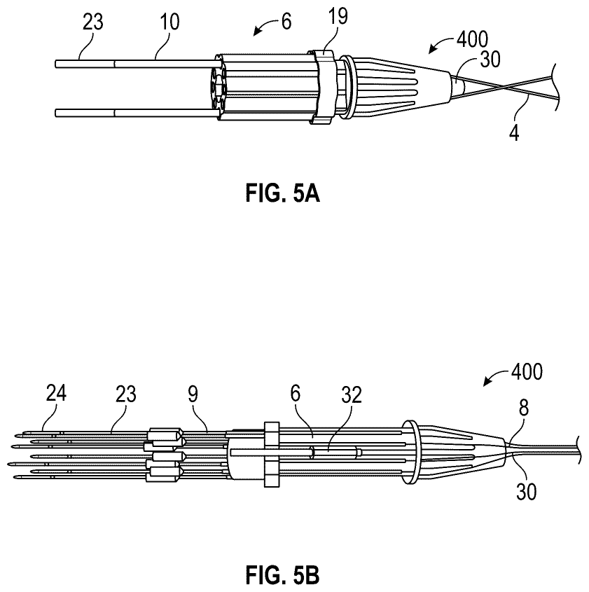

FIG. 5A is a perspective view of hub assembly.

FIG. 5B is a top view of the hub assembly.

The hub body 6 and hub cone 30 form the hub assembly. Pusher rods 9 attached to the proximal end of respective driving wires 4 extend from a proximal side of the hub assembly. Distal portions of the driving wires 4 extend from the distal side of the hub assembly. The drive wires 4 are slidable within respective hub hypotubes 8. As illustrated in the hub assembly of FIG. 5A, which omits the continuum robot, upon exit from the hub cone 30, the drive wires 4 naturally follow a path corresponding to a slope of the distal end of the hub cone 30. As illustrated in FIG. 5B, in which the continuum robot is included at a distal end of the hub assembly, the path that is naturally followed by the drive wires 4 is altered so that the drive wires extend in parallel along continuum robot. Although the push-pull assembly of FIG. 5B is longer than the push-pull assembly of FIG. 5A, each assembly requires similar components for pitch diameter transition. The hub hypotubes 8 of both FIG. 5A and FIG. 5B are similarly curved at the location due to the pitch diameter transition, which increases the risk of buckling. FIG. 5B illustrates unsupported sections of a plurality of pusher rods. The present disclosure provides a gradual transition from a larger, more rigid hub body 6 to a smaller, flexible catheter shaft 5.

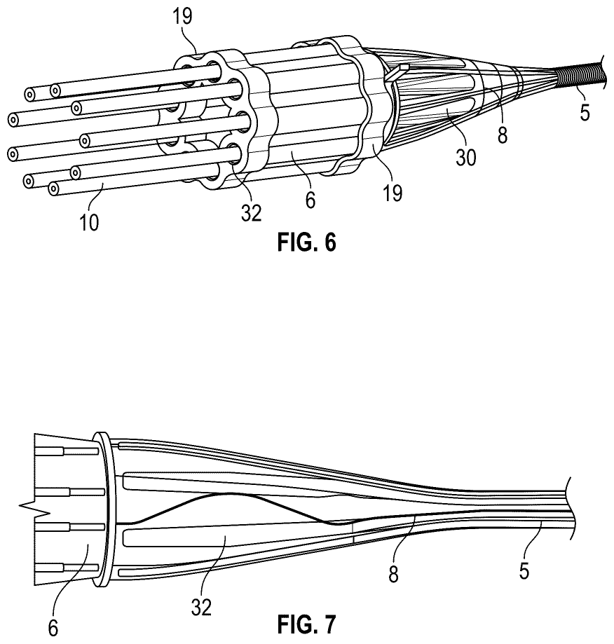

FIG. 6 is a rear perspective view of the hub body illustrating the single part hub body having multiple channels.

In contrast, the structure illustrated in FIG. 6 reinforces the pusher hypotube 10, to avoid damage to the pusher hypotube 10 based on forces exerted by the hypotube clamp sleeves 24 and clamp rods 23, as well as other sources of damage.

The hub body 6 of FIG. 6 may be formed as a single extruded part with a straight hub channel 32 for supporting respective push-pull assemblies. As shown in FIG. 6, hub guide disks 19 are provided at proximal and distal ends of the hub body 6. The hub disc 19 at the distal end may be used for attachment to the hub cone 30.

FIG. 7 illustrates hub hypotube buckling.

The hub hypotubes 8 illustrated in FIG. 7 lack support at the transition between pitch diameters. FIG. 7 illustrates buckling of the hub hypotube 8. Locations where the wire path curves, i.e., pitch diameter transitions, have increased risk of wire buckling. For a small diameter robotic catheter, the wire pitch in the catheter is smaller than that of the actuator pushing mechanism. In the hub cone 30, the wires will travel from a larger actuator pitch diameter through to a smaller catheter pitch diameter. For example, the wires may be clamped at a pitch diameter of 25 mm (due to the spacing of the clamps in the actuator), and the catheter shaft has a pitch diameter of 0.1225″ (3.115 mm).

The area of catheter-hub connection includes an attachment point and a curved wire path, continuous support of the drive wires avoids wire buckling. Thus, aspects of the present disclosure provide support for the hub hypotubes 8 in sections of pitch diameter transition to prevent buckling during pushing operation.

By way of non-limiting example, the steerable robotic catheter may include a small diameter (4 mm) catheter shaft 5 and hub hypotubes (0.018″ outer diameter (OD)), small (0.0095″) nitinol drive wires 4 configured to be pushed/pulled with a +/−16 mm stroke length with up to 20 N force, a wire pitch diameter increase from the catheter shaft to the actuator 2 from 3.1 mm to 22 mm. Particularly for small diameter hypotubes and long push stroke, hub hypotubes 8 may buckle within an unsupported section(s). Drive wires 4 may be 0.0095″ diameter nitinol wires; hub hypotubes 8 may be 304 SS 26TW, 0.012″ inside diameter (ID)/0.018″ OD, 120 mm length; pusher hypotubes 10 may be 304 SS 21RW, 0.020″ ID/0.032″ OD, 61 mm length; and a resilient member may be included with a 30 mm free length/9 mm solid length, 0.0025″ nitinol wire, 0.018″ OD.

FIG. 8 is a profile view illustrating components connecting to the catheter hub, according to the present disclosure.

FIG. 9 is a perspective view of the components connecting to the catheter hub, according to the present disclosure.

FIG. 10 is a perspective view of the proximal components connecting to the catheter hub, affixed in respective clamps of a controller, according to the present disclosure.

As shown in FIGS. 8 to 10, the tool channel 20 is provided that includes an exit port within the hub body 6. The tool channel 20 may be centrally located and may extend through the entire working length of the catheter 5, allowing utilization of tools, e.g., biopsy tools and endoscopes, during operation.

The tool channel 20 may enter the hub body 6 and extend through both the hub body 6 and the catheter shaft 5. The tool channel 20 may be bonded to the hub cone 30 and the catheter shaft 5. The tool channel 20 may have an OD of 0.091″/2.3 mm. The tool channel 20 may have a 2 mm ID to allow use of 1.8 mm biopsy tools. The tool channel 20 may be a 13XX stainless steel hypotube with 0.100″ OD/0.087″ ID to allow use of endoscopes and biopsy tools. A luer/pump attachment allows the tool channel 20 to be used for suction and irritation.

Tools may be inserted through the hub body 6 via the tool channel 20. Making the exit port of the tool channel 20 accessible from outside of the hub body 6 may require increasing the pitch diameter to create space between drive wires 4. An adapter, e.g., luer fitting, may be attached to the proximal end of the tool channel 20 outside the hub body 6 for connection to syringes, pumps and other instruments. A tube forming the tool channel 20 may be constructed of material that is sufficiently flexible to bend along the exit path while maintaining resistance to buckling when tools are pushed therethrough.

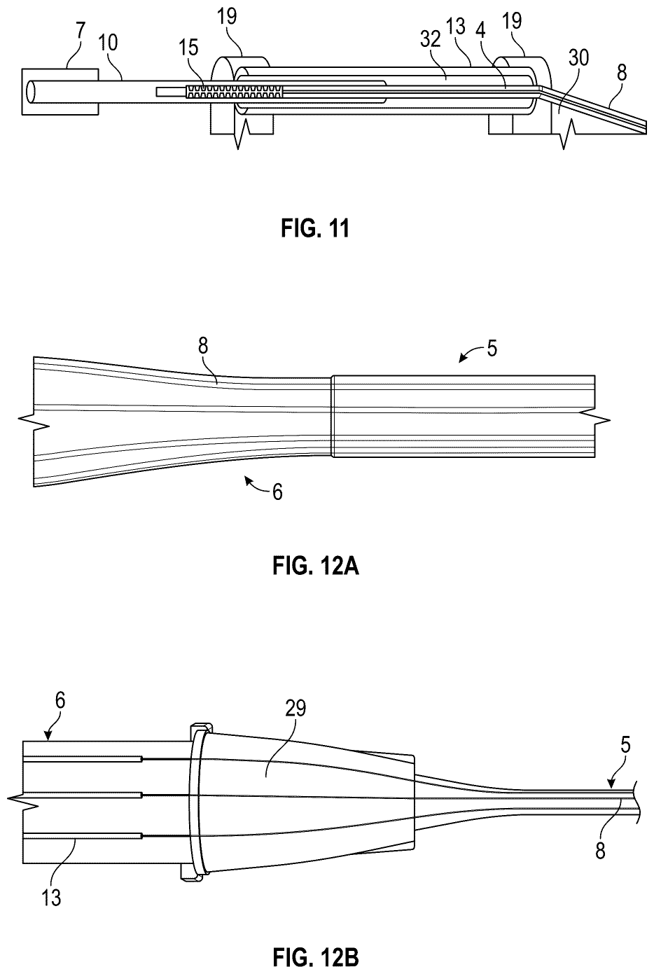

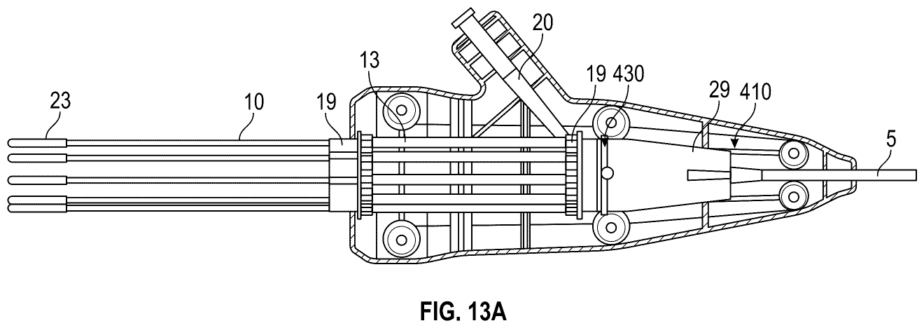

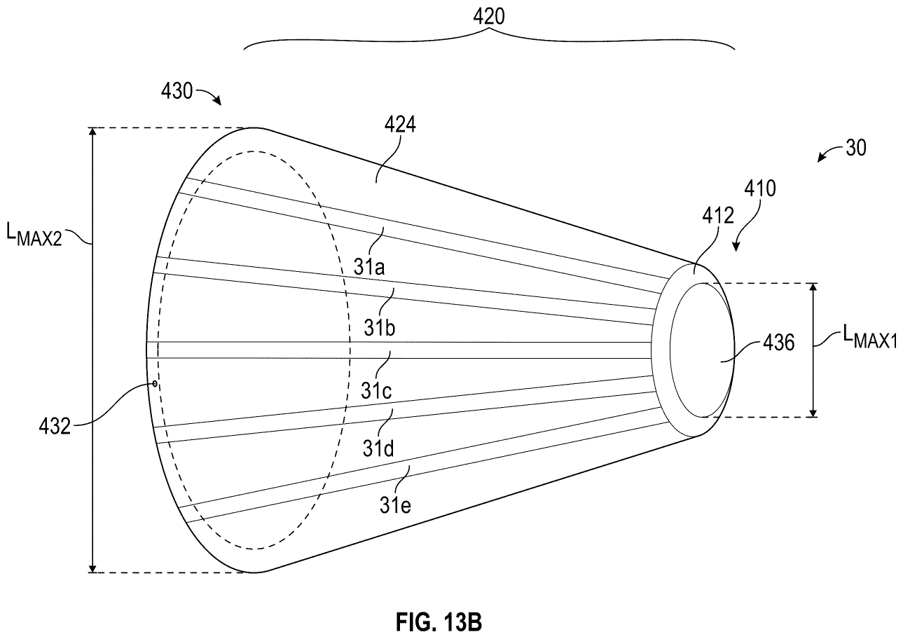

As illustrated in FIGS. 11 and 13A, the multi-part hub body may include a plurality of single lumen hub guide hypotubes 13 and guide disks 19, with the tool channel 20 exiting between the hub guide discs 19, with the catheter shaft 5 including a central lumen for tool passage through an entire working length thereof. The tool channel 20 may be inserted/bonded into the proximal end of the catheter shaft 5, to provide an inlet/outlet path for tools which are loaded from outside the hub body 6. An ID of the tool channel 20 may be sized to allow endoscopes and surgical tools to pass through without interference.

The steerable robotic catheter of the present disclosure supports the hub hypotubes and prevents drive wire buckling.

FIG. 11 is a cut away view of hub guide hypotubes and guide disks of the hub guide, according to the present disclosure.

As illustrated in FIG. 11, pusher hypotube 10 may be affixed to the controller via clamp 7. Deformable member 15 may be provided within pusher hypotube 10 to surround at least a portion of a respective driving wire 4. The deformable member 15 may be one or more of a foam, an elastomer, an elastic polymer, a thermoplastic, an unsaturated rubber, a saturated rubber, an organic rubber, and/or an inorganic rubber. The straight hub channel 32 is provided within the hub body 6 between hub guide disks 19. The pitch diameter transition may start on a distal side of the distal hub guide disk 19.

FIG. 12A illustrates an extended cone cover assembled in a pre-reflowed condition, according to the present disclosure.

FIG. 12B illustrates a cone cover assembled on the hub body in a reflowed condition, according to the present disclosure.

As illustrated in FIG. 12A, the hub 6 may abut the catheter shaft 5, with hub hypotubes 8 extending therethrough, prior to reflowing.

As illustrated in FIG. 12B, the hub cone 30 may be bonded onto the catheter shaft 5 with the hub hypotubes 8 transitioning therebetween. A proximal end of the catheter shaft 5 may include lumens that are larger than lumens on the distal end of catheter shaft 5. The larger lumens on the proximal end of the catheter shaft 5 are sized for an OD of the hub hypotube 8 housed therein. The distal ends of the hub hypotubes 8 may be inserted into proximal ends of the catheter shaft lumens. The smaller lumens on the distal end of catheter shaft 5 are sized for the ID of the drive wire 4 housed therein.

The cone cover 29 may cover the entire hub cone 6 and proximal end of the catheter shaft 5. The distal end of the cone cover 29 fits closely over the an OD of the catheter shaft 5. The proximal end of the catheter shaft 5 may have lumens 20 sized to accommodate an OD of the hub hypotube 8. The distal end of the hub hypotubes 8 is inserted into the proximal catheter shaft. The distal end of the tool channel 20 is inserted into a central tool channel lumen in the catheter shaft 5.

The cone cover 29, hub cone 6, tool channel 20, and catheter shaft 5 may all be formed of thermoplastic materials.

The hub cone 30, tool channel 20, catheter shaft 5, and hub hypotubes 8 may be thermally bonded, i.e., reflowed, together, as illustrated in FIG. 12B. Mandrels may be used to keep the tool channel and wire channels open, with the tool channel mandrel being a same diameter as the ID of the tool channel 20. The wire channel mandrels may be the same diameter as lumens of the catheter shaft 5 and the ID of the hub hypotube 8. As also illustrated in FIG. 12B, the hub 6 may be fixedly attached to the catheter shaft 5.

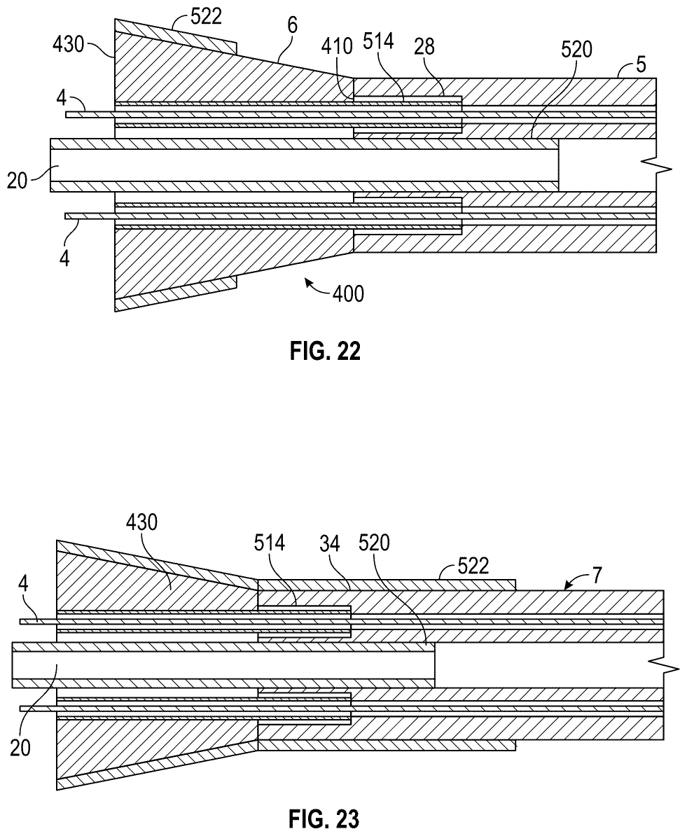

The catheter shaft 5 may have nine nitinol drive wires 4 with 0.0095″ OD. The hub hypotubes 8 may be 304 stainless steel, 26TW hypotubes, with 0.012″ ID/0.018″ OD. The tool channel 20 may be a single lumen 63D Pebax® extrusion, 0.091″ ID/0.104″ OD. The catheter shaft 5 may be a multi-lumen 72D Pebax® extrusion, 0.101″ ID/0.1461″ OD, with eighteen small lumens (nine used for drive wires) and a central lumen for tool passage, proximal catheter shaft lumen guide 28 (FIG. 22) may be 0.0165″ ID, and 5 mm length. Distal lumens of the catheter shaft 5 may be 0.0125″ ID, extending longitudinally through the distal bending section 102. The hub hypotubes 8 may be inserted 5 mm into the proximal catheter shaft lumen guide 28. The tool channel 20 may be inserted into the catheter shaft 5, ending 3 mm past the proximal edge of a distal end of the catheter shaft 5.

Locations where the path of the wires may curve increases the risk of buckling, such as the transition from a pushing mechanism at a proximal end of a hub body 6, with the transition being from a large diameter at a proximal end of a hub body 6 tapering to a smaller diameter at a distal end of a hub body 6 to match the smaller diameter catheter 5. Attachment points between the hub body 6, the catheter shaft 5, and other components may introduce ledges where wires or other slidable members may catch, due to, e.g., channel misalignment or gaps where surfaces are not flush or are unsupported. Transition between different materials may create friction. If parts are attached using adhesives, excess adhesive may enter the wire channel at these connection points during assembly. Assembly-wise, maintaining smooth wire transitions in an accurate and repeatable way may be difficult. For example, perfect alignment of support channels for multiple wires between two components is promoted by very accurate/repeatable assembly methods as well as parts having precise tolerance. Assembly variability may cause gaps where wires are unsupported outside of a catheter shaft extrusion. Due to risk of exposed wires being contaminated by glue, bonding the catheter extrusion to the tool channel or hub cone with adhesive is not performed, resulting in a weakened catheter-hub bond that may separate upon wire pushing. Since the tool channel 20 may not be bonded to the catheter shaft 5, the tool channel 20 may not be perfectly sealed/leakproof. The catheter 5 may also twist during assembly with the tool channel 20 and hub body 6, causing risk of misalignment. The assembly process is very time-consuming, due to the need to align each component and separately bond. Any assembly errors may result in inconsistent friction or other damage.

In the reflow process, the hub cone 30, tool channel 20, hub hypotubes 8, and catheter shaft 5 are reflowed together at ˜180 degrees C., with fluorinated ethylene propylene (FEP) heat shrink. The cone cover 29 may also be reflowed to the catheter-hub connection.

During the reflow process, the inner diameter of the tool channel 20 and catheter shaft 4 may be supported by a 0.091″ Polytetrafluoroethylene (PTFE) coated mandrel. The inner diameter of the hub hypotubes 8 and catheter lumens may be supported by 9x 0.0113″ PTFE coated mandrels, which are removed post-reflow. Thus, attachment of the hub body 6 to the catheter and manufacturability is improved. Regarding attachment of the hub body 6 to the catheter, reflowing the hub cone 30, tool channel 20, hub hypotubes 8, and catheter shaft 5 creates a strong attachment between the components in the limited bonding space. Regarding manufacturability, reflowing all parts together may be performed in a single manufacturing step, eliminating the individual bonding steps required for adhesive bonding. Reflowing provides a robust connection that is faster, easier to perform, and more repeatable than adhesive bonding, and reducing parts. After reflowing, the entire catheter-hub connection area is a solid structure conforming to all components, with no individual joints. Reflowing creates a gradual transition in material stiffness from the larger, rigid hub body 6 to the smaller, flexible catheter shaft 5, with improved stiffness and durability of the connection area. A smooth wire transition is provided to reduce friction and to reduce risk of buckling, with reflowing allowing the catheter shaft 5 material to re-form around the hub hypotubes 8 and the 0.0113″ mandrels, creating a smooth, uniform diameter channel for the drive wires 4 to slide through, without gaps or mismatched edges. The wire channel is also sealed from contamination, allowing use of suction/irrigation while preventing entry of dust/debris.

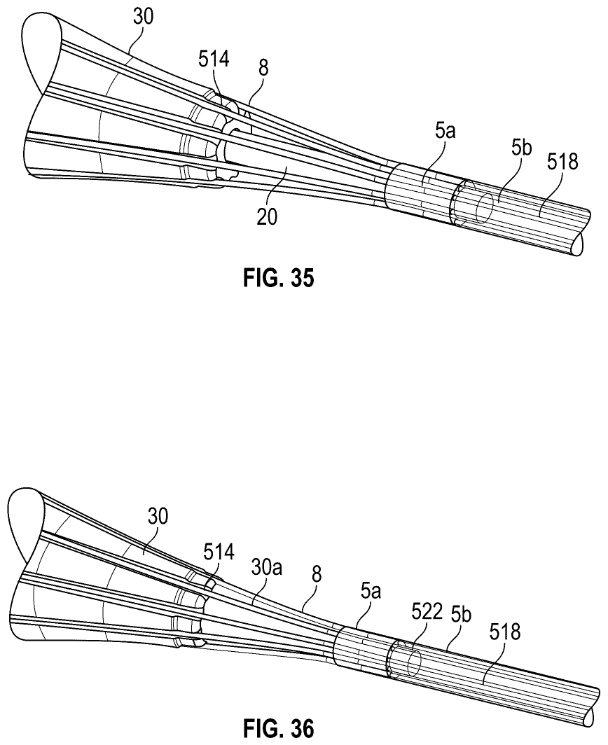

The reflow method attaches an inner cone 30a (FIG. 36), the hub cone 30, hub hypotubes 8, tool channel 20, and catheter shaft extrusions (5a and 5b, FIG. 35) together, without requiring expensive molds or reducing the need for precise machining of the hub cone 30. Attachment by the reflow method eliminates gaps between the hub body 6 and catheter shaft 5, reducing buckling of the hub hypotubes 8, buckling of the tool channel 20, catheter twisting/misalignment, and losses associated with flexing/moving of the hub hypotubes 8.

The reflow method provides a robust connection area by creating a solid, monolithic structure with a smoothly tapered outer diameter, which provides gradual stiffness transition from the hub body 6 to the catheter shaft 5. The reflowed cone shape of the hub body 6 provides strain relief and prevents damage to the catheter-hub connection area. Uniformity of manufacturing is also provided. Glue joints are eliminated and attachment points where failures may occur are reduced. Wire friction in the hub body 6 is reduced and the reflow method provides a gradual cone curve shape. Reducing the slope of the cone curve reduces wire friction in the hub cone and risk of wire buckling. Components including a proximal stiffener 520 (FIGS. 22, 23) may be connected/sealed in a single manufacturing step, in contrast to the multi-step process to individually attach the components. The reflowed material readily conforms to the exact shape of the assembly, and all components are maintaining in position while reflowing.

The cone cover 29 may closely fit over at least a first proximal curve on the hub cone 30, to securely press the hub hypotube 8 into a respective guide channel 31 when the cone cover 29 is affixed thereto. The cone cover 29 may be formed of thermoplastic styrene or other rigid material, e.g., Acrylonitrile Butadiene Styrene (ABS), with an inner diameter of 22.5 mm at the proximal end thereof. The cone cover 29 may be bonded to the hub cone 30 via a wicking adhesive.

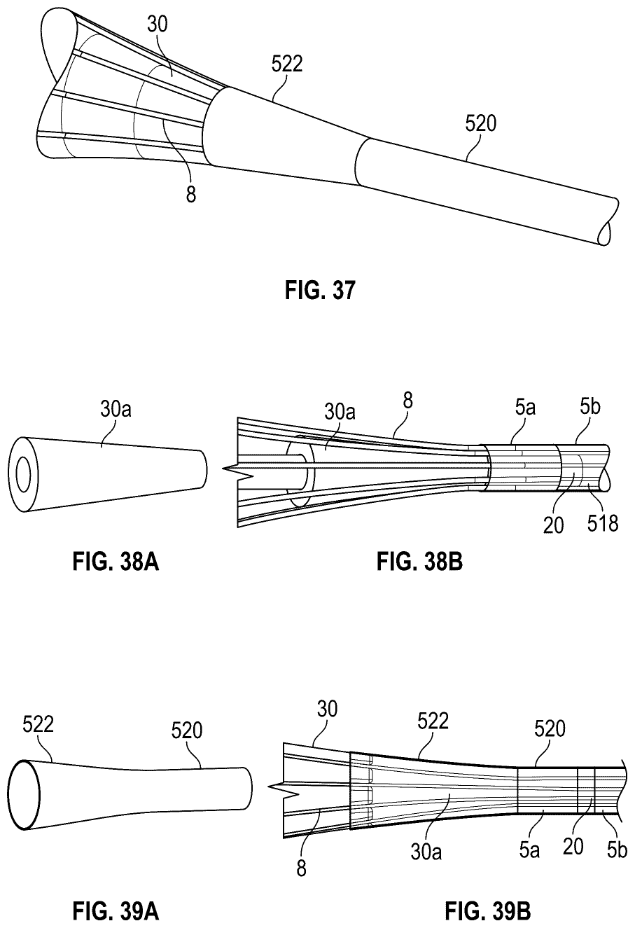

The cone cover 29 does not extend past a distal reflowed section of the hub cone 30 and a proximal flared stiffener 522 (FIG. 37, 40). By way of example, the hub cone 30 may end at a distance of 15 mm from the catheter shaft 5 (at ˜0.150″ pitch diameter). The inner cone 30a may have a flared section of a proximal stiffener 520 of 15 mm in length to fill a gap therebetween. An inner cone may be formed using stiffener material (e.g., 63D Pebax®). The inner cone 30a and proximal stiffener 520 may have a shape based on a shape of hub cone 30.

A gradual reduction in stiffness in the connection area is desired, from the larger/stiffer hub body 6 to the smaller diameter of the flexible catheter shaft 5. For catheter robustness, weak points of likely breakage/damage are avoided. Thus, reflowing may be utilized for construction.

The tube forming the tool channel 20 may be single lumen 63D Pebax® extrusion having a 0.101″ OD and 0.091″ ID. A 0.091″ PTFE coated stainless steel (SS) mandrel may be used to support the ID of the tool channel 20 during reflow. The tool channel 20 may be reflowed ˜3 mm into the distal catheter shaft. PTFE coated SS mandrels used to keep the lumens of the distal catheter shaft lumens 148 open during reflow may be 0.113″. Smooth wire transition is provided with a fully sealed tool channel 20 that is usable for suction and irrigation. Also, smooth, edge-free transitions are obtained to avoid catching working tools. The reflowed transition from the support sleeves to catheter lumens smooth with a uniform diameter channel. Thus, wire buckling and friction during wire movement is reduced. The reflowed assembly bonds all components in a single step. In contrast, conventional assembly takes at least four or more steps, i.e., gluing individual support sleeves into catheter lumens, tool channel to catheter shaft, catheter shaft to hub cone, and tool channel to hub cone. Reflowing extrusions is more cost-effective than other methods to create a single extrusion with two different sized lumens. Repeatability and accuracy of assembly is promoted, where all components being assembled together and held in place with a fixture during reflow to ensure correct alignment. Conventional assembly methods that use adhesives to bond components are difficult to control due to wicking of adhesives into lumen(s)/wire channels. Adhesive processes are also time-consuming due to allow proper cure times, which are often several hours. The reflowed structure conforms to an exact desired assembly shape, which eliminates misalignment and bond strength failures at attachment points.

The clamp rod 23 includes a hollow extending through a longitudinal length thereof, with an opening through which a proximal end the pusher hypotube 10 may be inserted, for the clamp rod 23 to fully cover the pusher hypotube 10. An ID of the clamp rod 23 closely fits over an OD of the pusher hypotube 10. The clamp rod 23 may be bonded to the distal end of the pusher hypotube 10. The clamp rod 23 may be removably affixed to the actuator clamp 7. The proximal end of the pusher hypotube 10 may be shortened to end after a wire attachment location. The clamp rod 23 may be constructed of, or coated with, an electrically insulating material. A distance from the distal end of the pusher hypotube 10 to the actuator clamp 7 and a distance from the proximal end of the pusher hypotube 10 to the actuator clamp 7 may be longer than distances for creepage/clearance, to electrically isolate the pusher hypotube 10 from the actuator clamp 7.

The pusher hypotube 10 may be a 21RW hypotube (0.032″ OD/0.020″ ID), 61 mm length. The 61 mm length may include 10 mm for the wire attachment, 30 mm for a deformable member 15 (FIG. 11), and 21 mm for a pull stroke length and minimum overlap length. The deformable member 15 may surround at least a portion of the at least one driving wire 4 extending through the support sleeve and may be one or more of a foam, an elastomer, an elastic polymer, a thermoplastic, an unsaturated rubber, a saturated rubber, an organic rubber, and/or an inorganic rubber.

The clamp rod 23 may be a clear polycarbonate tube, with 1.0 mm ID/2.5 mm OD, 91 mm length. The pusher hypotube 10 may be bonded to the clamp rod 23 with Loctite 4311 UV adhesive. The clamped length of the actuator clamp 7 may be 20 mm from the proximal end of the clamp rod 23. Creepage distance for electrical isolation between the catheter drive wire 4 and the actuator clamp 7 may be 4.0 mm, and the clearance distance may be 2.5 mm. The proximal end of the clamp rod 23 may be filled with adhesive, and the distance between the distal edge of the actuator clamp 7 and the distal end of the clamp rod 23 may be 71 mm, to exceed the creepage/clearance requirement. As can be appreciated, the dimensions provided herein are exemplary and may be modified to accommodate other pathways or desired points of interest in a given circumstance.

FIG. 13A is a cutaway profile view of the hub body, according to the present disclosure.

As illustrated in FIG. 13A, a plurality of pusher hypotubes 10 extend from a proximal end of the hub body 6 to respective clamp rods 23. A drive wire 4 provided within each pusher hypotube 10. Each pusher hypotube 10 transitions into the hub body 6 through a proximal hub guide disk 19. The drive wires 4 traverse the hub body 6 in respective hub hypotubes 8, exiting through a distal hub guide disk 19. Spacing between the hub hypotubes 8 reduces as the hub hypotubes 8 transition from a distal end of the distal hub guide disk 19 through the hub cone 30 to the shaft 5. Drive wires 4 exit respective hub hypotubes 8 at or along the catheter shaft 5.

An outer shell 34 may be a two-part shell secured together by screws, snaps, or similar components. When assembled, the outer shell 34 may cover the hub body 6 the assembled hub cone 30 and cone cover 29. The hub cone 30, hub hypotubes 8, and the catheter shaft extrusions (5a and 5b, FIG. 35) may be compressed together by the assembled outer shell 34 for connection thereof.

FIG. 13B is a view of the hub cone, according to the present disclosure.

As illustrated in FIGS. 13A and 13B, an aspect of the present disclosure provides a hub cone 30 that includes a distal end 410 with a first face 412, a proximal end 430 with a second face 432, and a section 420 extending between the first face 412 and the second face 432.

The first face 412 has a first maximum length Lₘₐₓ₁ between sides thereof. The second face 432 has a second maximum length Lₘₐₓ₂ a between sides thereof. The second maximum length Lₘₐₓ₂ is larger than the first maximum length Lₘₐₓ₁.

The first face 412 and the second face 432 may be substantially round, elliptical, and/or quadrilateral in shape. When the face 412 is substantially round or elliptical in shape, the first maximum length Lₘₐₓ₁ is a first diameter, section 420 is substantially conical, and the second maximum length Lₘₐₓ₂ is a second diameter. When the face 412 is substantially quadrilateral in shape, the first maximum length Lₘₐₓ₁ is a longest distance between sides of the substantially quadrilateral shape along the first face 412, and the second longest length Lₘₐₓ₂ is a longest distance between sides of the substantially quadrilateral shape along the second face 432.

Section 420 of the hub cone 30 extends from the distal end 410 to a proximal end 430. A hollow 436 extends through section 420 from the first face 412 to the second face 432. A plurality of hub guide channels 31a, 31b, . . . 31e extend along at least a part of a longitudinal length of an exterior surface 424 of section 420.

ODs of the hub hypotubes is smaller than IDs of hub insert tubes, so that the hub hypotubes fit within respective hub insert tubes. The hub insert tubes are substantially longitudinal, so that the hub insert tubes straighten the portion proximal end of the hub hypotubes 8 that are inserted therein. The hub insert tubes are substantially parallel to the hub guide tubes 18, and are supported by the guide disk 19. The hub insert tubes are positioned with at least one push stroke distance from a proximal end of the respective clamp rod 23.

As shown in FIG. 13A, a tool channel 20 may be provided in the hub cone 30. At least a part of an inner surface of the outer shell 34 fits closely to at least a part of an outer surface of the hub cone cover 29, and the outer shell 34 may support an exit port of the tool channel 20.

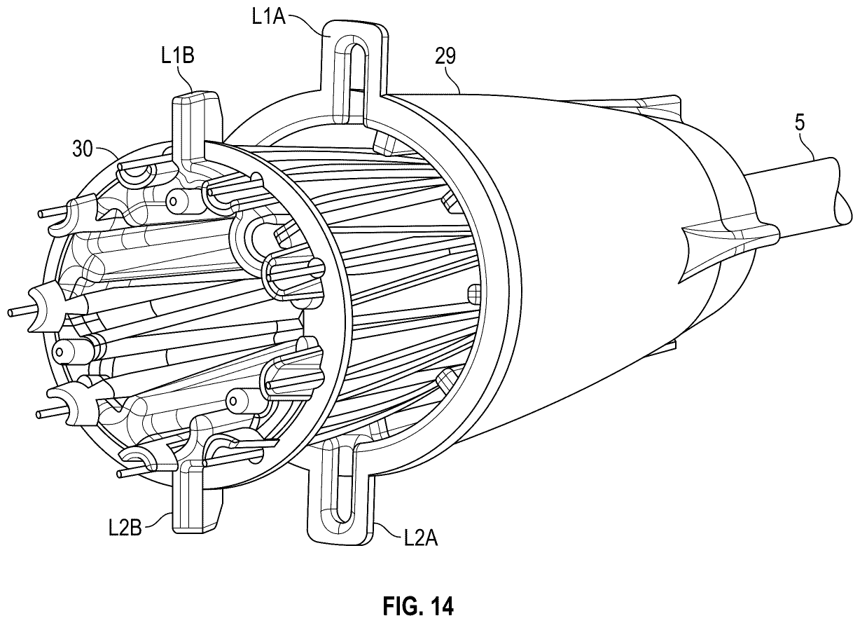

FIG. 14 is a perspective view of the hub cone with a cone cover disassembled therefrom, according to the present disclosure.

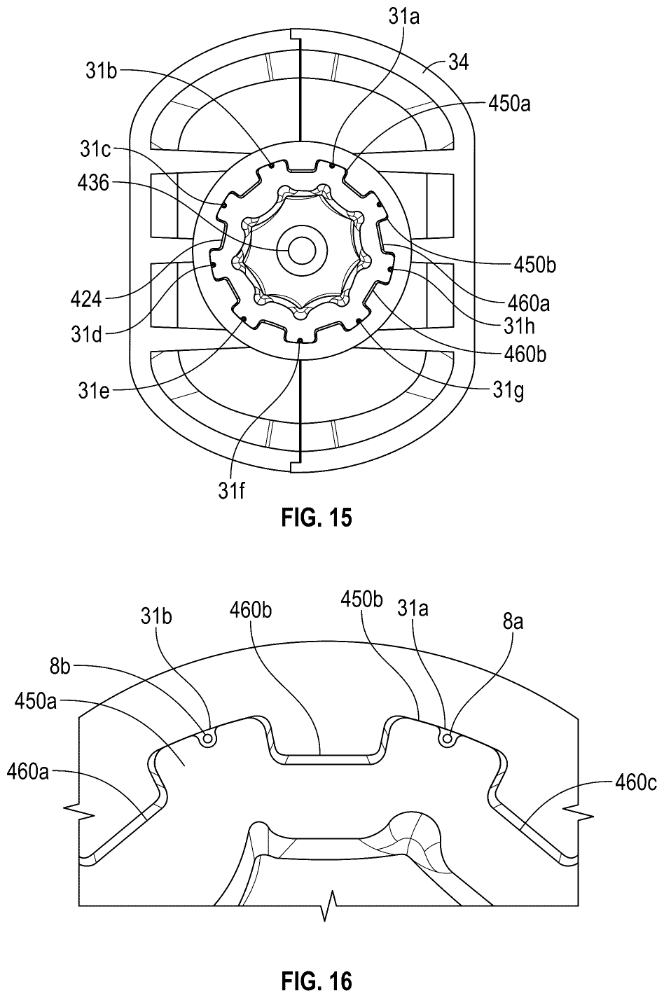

In FIG. 14, the hub cone 30 and cone cover 29 are viewed from a proximal end, with the hub cone 30 partially removed/disassembled from the cone cover 29. When assembled onto the hub cone 30, the cone cover 29 prevents the hub hypotubes 8 that are within hub guide channels 31a . . . 31h (FIG. 15) from moving away from/buckling the hub cone 30 when a pushing force is exerted on an end of one or more of the drive wires 4. The hub hypotubes 8 may be a rigid material, e.g., 26TW SS, with an inner diameter of 0.012″ and an outer diameter of 0.018″. The outer cone cover 200 may be molded TPU and the hub cone 30 may be molded 72D Pebax®.

The cone cover 29 and the hub cone 30 may include interlocking grooves, as shown in FIG. 14, for alignment and to prevent twisting of the cone cover 29 withing the hub cone 30.

The cone cover 29 may lock onto the proximal portion of the hub cone 30. Locking may be performed by engaging tab L_{1B} that is provided on a circumference of the hub cone 30 into a correspondingly sized opening in a receiving tab Lia provided on a circumference of the cone cover 29. As shown in FIG. 14, more than one engaging tab L_{1B} and receiving tab Lia may be provided on the hub cone 30 and the cone cover 29, respectively. Thus, the cone cover 29 prevents the hub hypotubes 8 from moving or buckling when the wires are pushed, and prevents twisting of the cone cover 29 withing the hub cone 30. The cone cover 29 may be removed/replaced by reversible attachment to the hub cone. The cone cover may be used to hold the hub hypotubes 8 in place for the reflow process.

As shown in FIG. 13A and FIG. 14, the outer shell 34 may enclose the hub cone 30, the hub cone cover 29, and the plurality of hub hypotubes 8. When attached to the hub cone 30, the cone cover 29 presses the hub hypotubes 8 against the exterior surface 424 of the hub cone 30.

FIG. 15 is a cutaway profile view of the hub cone 30, cone cover 29 and outer shell 24, according to the present disclosure.

FIG. 16 is an expanded view of FIG. 15.

As shown in FIGS. 15 and 16, a plurality of hub guide channels 31a, 31b, . . . 31e may be provided on the exterior surface 424 of the hub cone 30 around the hollow 436. The plurality of hub guide channels 31a, 31b, . . . 31e are configured to receive a respective hub hypotube 8a, 8b therein. The hub cone 30 may include a plurality of depressions 460a, 460b, 460c that extend along the longitudinal length of the exterior surface 424 of the section 420. Each depression of the plurality of depressions 460a, 460b, 460c is positioned between a respective pair of guide channels of the plurality of hub guide channels 31a, 31b, . . . 31e.

The hub cone 30 may also include a plurality of plateaus 450a, 450b and a plurality of depressions 460a, 460b, 460c. Each depression of the plurality of depressions 460a, 460b, 460c and each plateau of the plurality of plateaus 450a, 450b extends along the longitudinal length of the exterior surface 424. Each hub guide channel of the plurality guide channels 31a, 31b, . . . 31e is positioned between a respective pair of plateaus of the plurality of plateaus 450a, 450b. Each depression of the plurality of depressions 460a, 460b, 460c is positioned between a respective pair of hub guide channels of the plurality of hub guide channels 31a, 31b, . . . 31e.

Each hub hypotube 8 may be positioned in a respective guide channel of the plurality guide channels 31a, 31b, . . . 31e. As such, a portion of an outer surface of each hub hypotube 8 protrudes from the exterior surface 424 of the hub cone 30.

The cone cover 29 may be compressed by the assembled outer shell 34, which surrounds the entire hub body 6, the hub cone 30, and an area connecting the distal end of the hub body 6 to the catheter shaft 5. The cone cover 29 may be formed of a soft, compressible material, and the outer shell 34 may be slightly undersized to compress an outer surface of the cone cover 29 when assembled onto the cone cover 29. Such assembly simplifies assembly and manufacture, since all of the hub hypotubes 8 may be secured against the hub cone 30 by closing the outer shell 34 onto the cone cover 29. Such assembly also eliminates buckling of hub hypotubes 8. The 26TW that may be utilized for the hub hypotubes 8 may have a small diameter, e.g. 0.46 mm, relative to the hub body 6. The cone cover 29 may cover the hub cone 30 and a proximal extrusion of the catheter shaft 5. The cone cover 29 may extend at least partially over a distal extrusion of the catheter shaft 5. The cone cover 29 may fit closely onto a distal portion of the catheter shaft 5 and has a tapered distal end. The cone cover 29 may be formed of an elastomer having a durometer scale lower that a durometer scale of the plurality of hub hypotubes 8 and the hub cone 30. When wrapped around at least part of the exterior surface of the hub cone 30, the hub cone cover 29 presses the hub hypotubes 8 against the hub cone 30 to seal the hub hypotubes 8 within the hub cone 30 and the hub cone cover 29.

The cone cover 29 may slide or be rolled over a proximal end the hub cone 6, with the hub hypotubes 8 assembled thereon. When connected by reflow, the hub cone 30, tool channel 20, catheter shaft 5, and hub hypotubes 8 are thermally bonded. The cone cover 29 may be formed of a thermoplastic material and may be reflowed to the hub cone 30, hub hypotubes 8, tool channel, and the catheter shaft extrusion, and thermally bonded. The cone cover 29 need not be attached to the hub cone 30, providing ease of assembly and fewer components.

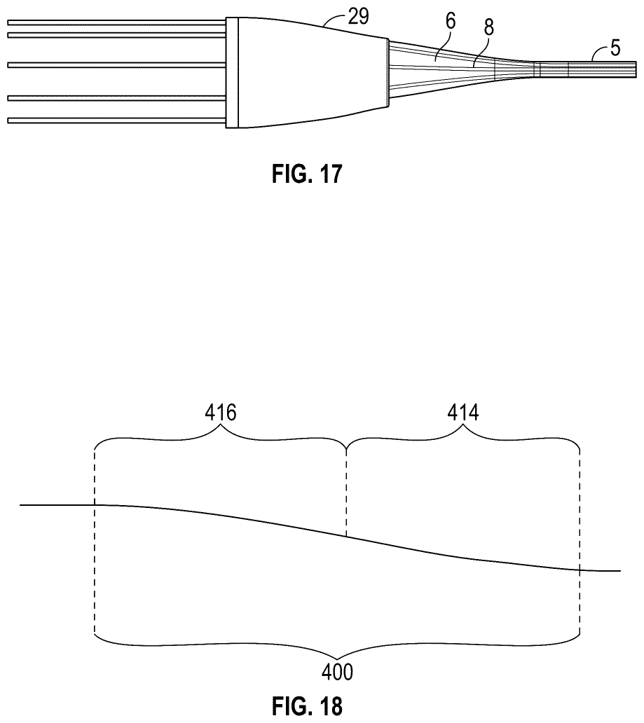

FIG. 17 is a side view of a hypotubes, a cone cover, a hub body and catheter shaft, according to the present disclosure. The hub cone 30 may include a plurality of hub guide channels 31a, 31b, . . . 31e (FIG. 13B) that extend along the longitudinal length of the exterior surface 424 of the section 420. The plurality of hub guide channels 31a, 31b, . . . 31e may be located between the hub cone 30 and the cone cover 29. Each hub guide channel of the plurality of hub guide channels 31a, 31b, . . . 31e may be configured to accommodate at least a portion of a hub hypotube therein.

As shown in FIG. 17, the plurality of hub hypotubes 8 extend between the hub cone cover 29 and the hub cone 30. The hub cone cover 29 may be configured to cover a second section of the hub cone 30.

FIG. 18 shows changes in pitch diameter across the hub cone, according to the present disclosure.

FIG. 18 is a partial side view of the hub cone 30 illustrating a first pitch diameter change and a second pitch diameter change that occurs across a longitudinal length of the hub cone 30. The first pitch diameter change extends from the distal end 410 of the hub cone 30 along a first length 414 of section 420 of the hub cone 30. The second pitch diameter change extends from an end of the first pitch diameter change along a second length 416 to the proximal end 430 of the hub cone 30. The first length 414 is longer than the second length 416. The first pitch diameter change defines a substantially concave curve. The second pitch diameter change defines a substantially convex curve.

The pitch diameter change illustrated in FIG. 18 generally conforms to a natural path of the hub hypotubes 8 (26TW SS hypotubes) starting from a 22 mm pitch diameter at the hub body 6 and tapering to a 0.1225″ pitch diameter at the catheter shaft extrusion (5a and 5b, FIG. 35), across a 70 mm cone length. Lengthening the hub cone 30 and using the natural curve path of the hub hypotubes 8 may reduce wire friction within the hub cone 30, by smoothing the wire path for a more gradual transition. Additionally, the natural curve shape improves ease of assembly. When assembling all components to be reflowed, the hub hypotubes 8 fit naturally without the need for a fixture.

As shown in FIGS. 17 and 18, the hub cone cover 29 may support the hub hypotubes 8 only along the substantially concave curve of the first length 414 of the hub cone 30. The hypotubes 8 that extend from the proximal end 430 of the hub cone 30 align with respective clamps 7 (FIG. 10) of the actuator 2 (FIG. 1), thereby eliminating bends in the hypotubes 8 between the hub body and the actuator 2. The hypotubes 8 that extend from the distal end 410 of the hub cone 30 align with the catheter shaft 5. Thus, when the continuum robot is in a relaxed mode, the hypotubes 8 within the shaft are substantially parallel to each other.

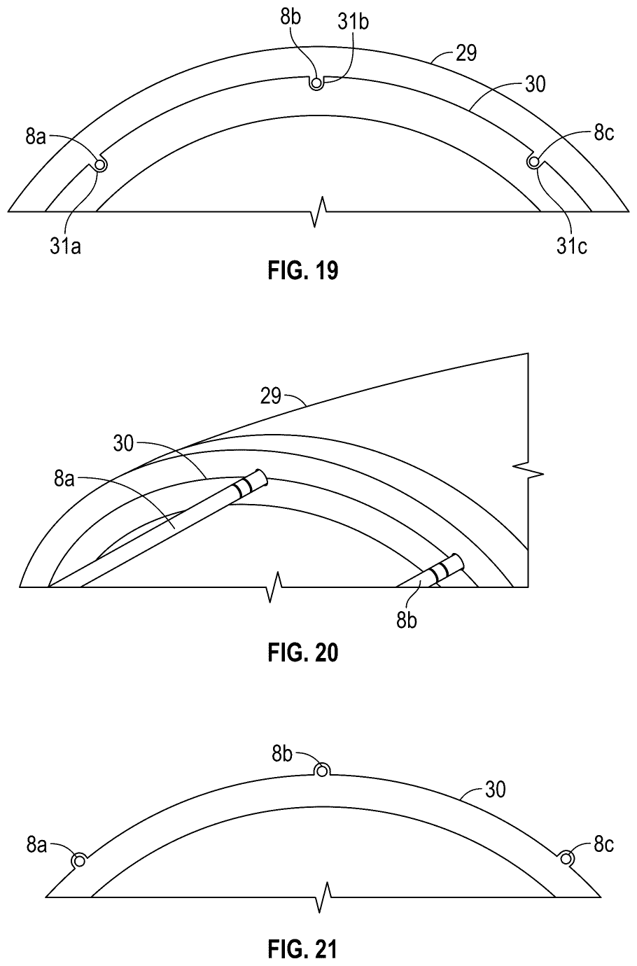

FIG. 19 is a cut away view illustrating hub hypotube positioning within a catheter shaft, according to the present disclosure.

As shown in FIG. 19, a plurality of hub guide channels 31a, 31b, 31c are provided that each extend along the longitudinal length of the exterior surface 424 of the hub body 6. Each hub guide channel of the plurality of hub guide channels 31a, 31b, 31c is configured to receive a respective hub hypotube 8a, 8b, 8c therein. As illustrated in FIG. 19, the depth of each hub guide channels 31a, 31b, 31c may substantially correspond to the OD of the respective hub hypotubes 8a, 8b, 8c so that an outer surface of each hub hypotube 8a, 8b, 8c is flush with the exterior surface 424 of the hub cone 30. For clarity, only three hub guide channels 31a, 31b, 31c and respective hub hypotube 8a, 8b, 8c are illustrated in FIG. 19, but the present disclosure is not limited to three.

FIG. 20 is a perspective view illustrating positioning of hub hypotube within a catheter shaft, according to the present disclosure.

As illustrated in FIGS. 19 and 20, each hub hypotube 8 of the plurality of hub hypotubes may be substantially round in shape, and each hub guide channel of the plurality of hub guide channels 31a, 31b, . . . 31e may be substantially round in shape, with a diameter that is equal to or larger than a diameter of the hub hypotube that is accommodated therein. The plurality of hub guide channels 31a, 31b, . . . 31e may be configured to accommodate respective hub hypotubes 8 therein. The plurality of hub guide channels 31a, 31b, . . . 31e and the respective hub hypotubes 8 may be located between the hub cone 30 and the cone cover 29.

FIG. 21 is a cut away view illustrating hub hypotube positioning within a catheter shaft, according to another embodiment.

In contrast to FIG. 19, the embodiment of FIG. 21 provides hub guide channel channels 31a, 31b, . . . 31e that are shallower and may only accommodate only a portion of a hub hypotube therein. Thus, at least a portion of the outer surface of each hub hypotube 8a, 8b, 8c protrudes from the exterior surface 424 of the hub cone 30.

FIG. 22 is a cut away view of a connection between the catheter and the hub, according to the present disclosure.

As illustrated in FIG. 22, the hub body 6 may abut the shaft 5.

As shown in FIG. 22, a distal end of the hub cone 30 includes the proximal end 430 of the pitch diameter transition 400, with drive wires 4 and the tool channel 20 extending through the hub body 6 to the shaft 5. The flared end 522 of the proximal stiffener 520 is provided at the distal end 410, with support sleeve guide channels 514 extending therethrough.

FIG. 23 is a perspective view of an extended cone cover, according to the present disclosure.

FIG. 23 is similar to FIG. 22, with the flared proximal end 522 extending beyond the proximal stiffener 520.

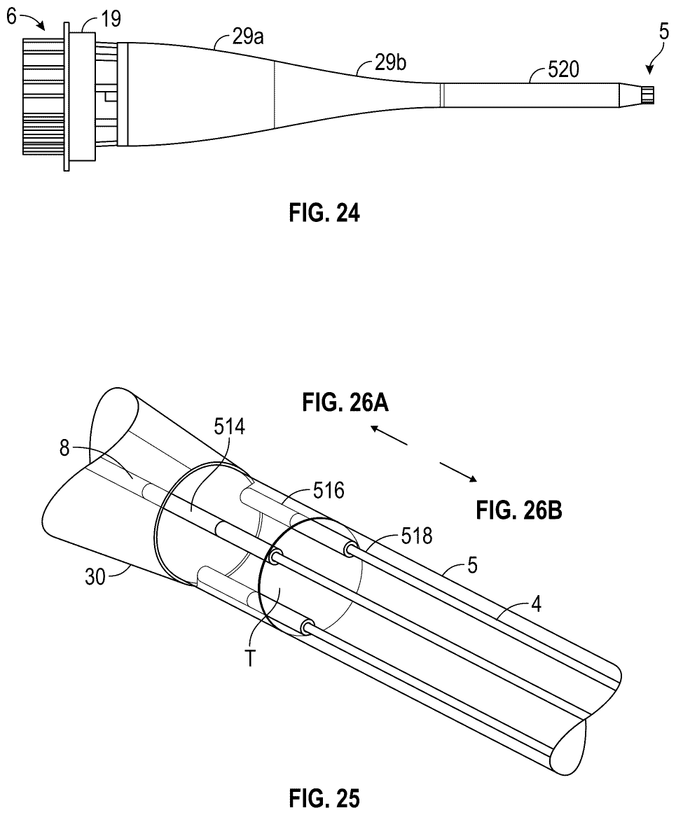

FIG. 24 is a perspective view of a hub hypotube sleeve connection with the shaft, according to the present disclosure.

As shown in FIG. 24, the cone cover 29 may extend over the entire hub cone 6 and partially cover a proximal end of the catheter shaft 5, extending to a proximal stiffener 520. The cone cover 29 may include a proximal part 29a and a distal part 29b. A proximal stiffener 520 may extend from the distal part 29b of the cone cover 29. Drive wires 4 extend in respective hypotubes from the hub body 6, through the hub guide disk 19, the proximal part 29a, the distal part 29b, and the shaft protrusion 55, to the catheter shaft 5. The shaft 5 may be a multi-lumen extrusion, with a respective lumen provided for each drive wire 4.

The hub body 6 transitions from a proximal wire pitch diameter that substantially aligns with the actuator clamps 7 to a distal wire pitch diameter that substantially aligns with the diameter of the catheter shaft 5. The change of wire pitch diameters, i.e., the pitch diameter transition, is followed by the hypotubes 8, i.e., lumens, as well as the drive wires 4 that are slidably provided therein, that extend from the controller 50 to the catheter shaft 5.

FIG. 25 is a perspective partial cut away view of a transition from a distal end of the hub cone to a proximal end of the catheter shaft, according to the present disclosure.

As shown in FIG. 25, hub hypotubes 8 extending from the distal end of the hub cone 30 may extend through support sleeve guide channels 514, support sleeve lumens 516 and drive wire lumens 518.

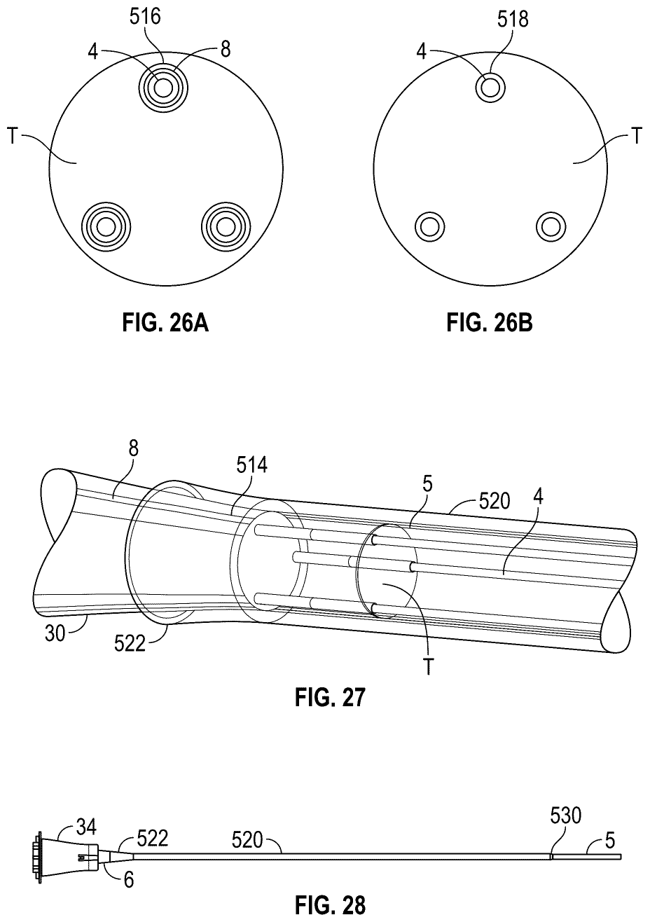

FIG. 26A is a cut away view at a transition between the support sleeve lumens and the drive wire lumens, according to the present disclosure.

FIG. 26B is a reverse view cut away view from FIG. 26a, according to the present disclosure.

The drive wires 4 extend from the hub body 6 to the catheter shaft 4. As illustrated in FIGS. 26a and 26b, the hub hypotubes 8 on the hub body 6 side extend to a transition T, but do not extend throughout the entire catheter shaft 5. As illustrated in FIG. 26A, the support sleeve lumens 516 surround the hub hypotubes 8 from within at least a distal part of the hub body to the transition T. From transition T toward the distal end of the catheter 5, the drive wires 4 are surrounded and protected by respective drive wire lumens 518. As illustrated in FIGS. 25 and 26A, a proximal end of the support sleeve lumens 516 may be bonded to at least a portion of the transition T.

FIG. 27 is perspective partial cut away view of a transition from a distal end of the hub cone to a proximal end of the catheter shaft, according to another embodiment.

As shown in FIG. 27, a flared proximal end 522 of the proximal stiffener 520 is provided for the distal end 410 to fit within.

FIG. 28 is a view of components extending from the outer shell to a proximal end of the catheter shaft 5, according to the present disclosure.

As shown in FIG. 28, the hub body 6 extends from a distal end of the outer shell 34, with the flared proximal end 522 and the proximal stiffener 520 ending to a tapered distal end 530 of the proximal stiffener 520.

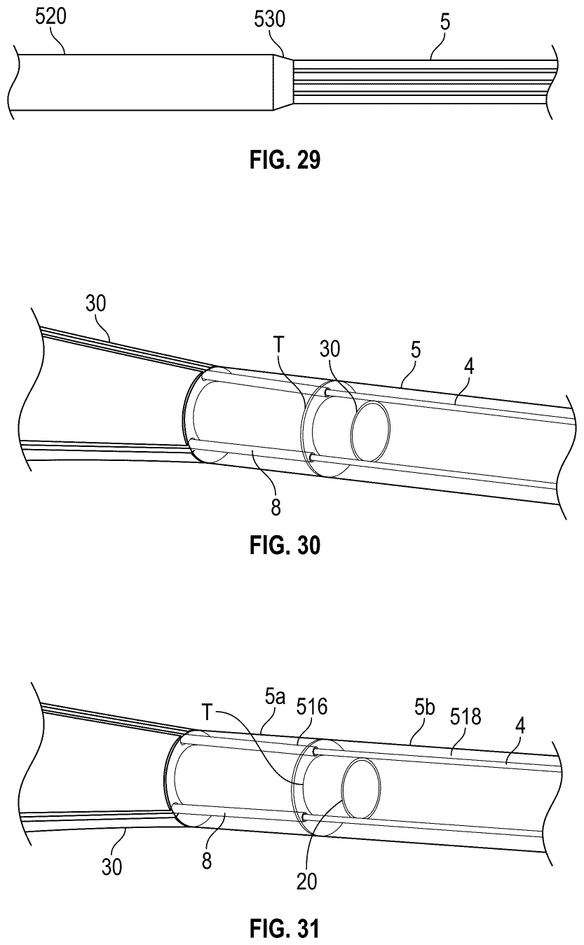

FIG. 29 is a detailed view of the tapered distal end of the proximal stiffener, accordingly to an embodiment.

As shown in FIG. 29, a distal end of the tapered distal end 530 of the proximal stiffener 520 closely surrounds and supports the catheter shaft 5.

FIGS. 30 and 31 are perspective partial cut away views of a transition from a distal end of the hub cone to a proximal end of the catheter shaft, according to another embodiment.

As shown in FIG. 30, the tube for the tool channel 20 may extend to an area distal of the transition T. FIG. 31 provides a rotated view of the tube for the tool channel 20 extending to the area distal of the transition T. For conciseness, FIGS. 25, 26, 30 and 31 illustrate three driving wires 4. However, the present disclosure is not limited to only three driving wires and may include at least nine driving wires 4.

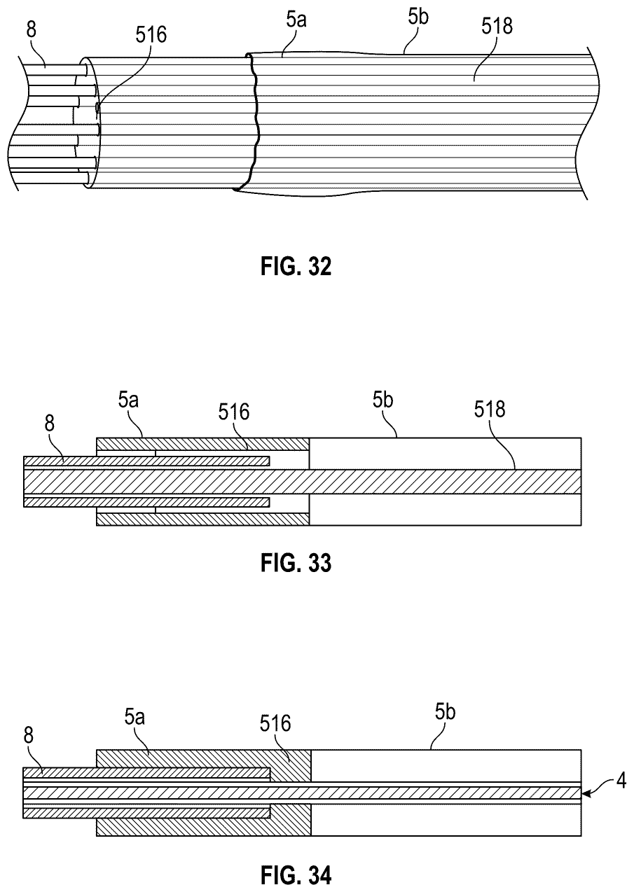

FIG. 32 is a perspective view of a reflowed proximal stiffener, accordingly to an embodiment.

FIG. 33 is a cut away view of components adjacent to the support sleeve lumens 516, before reflow, according to the present disclosure.

FIG. 34 is a cut away view of components adjacent to the support sleeve lumens 516, after reflow, according to the present disclosure.

As shown in FIGS. 33 and 34, the hub hypotube 8, proximal catheter shaft 5a and distal catheter shaft 5b are adjacent to the support sleeve lumens 516.

As shown in FIG. 32, a gap is present between the distal end of the hub hypotube 8 and the distal catheter shaft 5b, pre-reflow. As shown in FIGS. 32 and 34, reflow eliminates the gap.

FIGS. 35 and 36 are perspective partial cut away views of a transition from a distal end of the hub cone to a proximal end of the catheter shaft, according to another embodiment.

As shown in FIG. 35, before reflow a gap is present between the support sleeve guide channels 514. As shown in FIG. 36, after reflow, the inner cone 30a is formed between the support sleeve guide channels 514, in place of the gap.

FIG. 37 shows a proximal flared stiffener positioned on a proximal side of the proximal stiffener, according to the present disclosure.

As shown in FIG. 37, the proximal flared stiffener 522 surrounds the drive wires 4 and hypotubes 8, between the cone 8 and the proximal stiffener 520.

FIGS. 38A and 38B show assembly of the inner cone 30a, according to the present disclosure.

As shown in FIGS. 38A and 38B, the inner cone 30a may have a flared section that conforms to an inner area between the hub hypotubes 8, with a hollow for the tool channel 20.

FIGS. 39A and 39B show the proximal flared stiffener and the proximal stiffener, according to the present disclosure.

As shown in FIGS. 39A and 39B, a proximal flared stiffener 522 may have a shape that conforms to the inner area between the hub hypotubes 8, with a hollow for the tool channel 20, and may be positioned between the hub hypotubes 8, with a hollow for the tool channel 20.

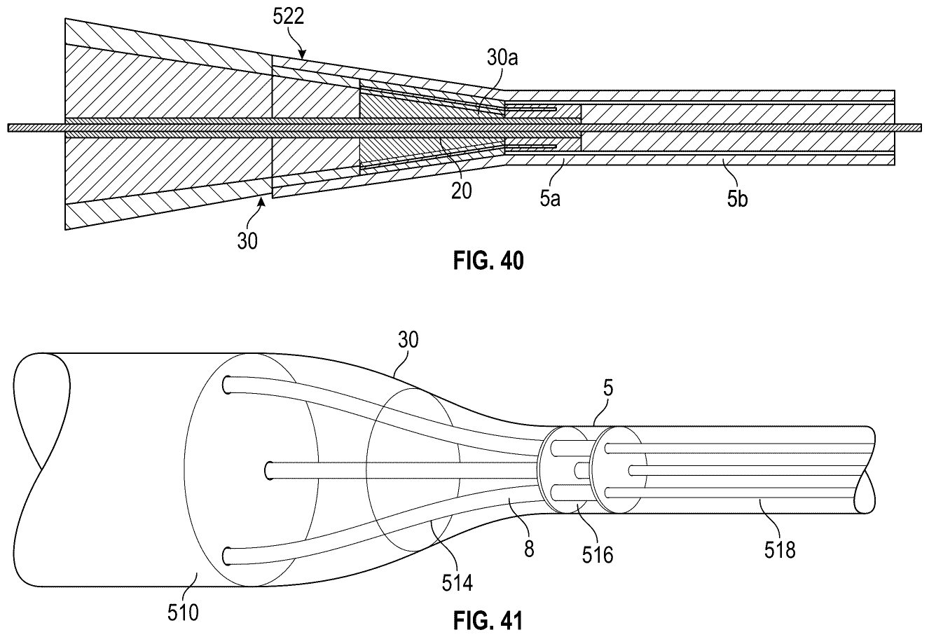

FIGS. 40 and 41 show the tool channel extending from the hub cone through the catheter shaft, according to the present disclosure.

As shown in FIG. 40, the inner cone 30a is positioned between the hub cone 30 and the proximal catheter shaft extrusion 5a. As shown in FIG. 41, the hub hypotubes 8 extend from the distal end of the hub cone 30 through support sleeve guide channels 514, support sleeve lumens 516 and drive wire lumens 518.

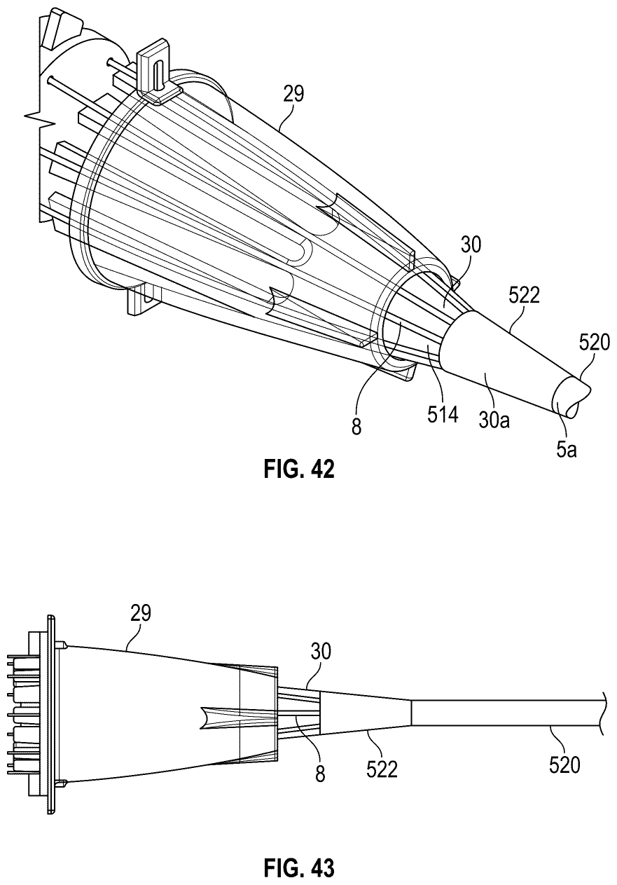

FIG. 42 is a perspective view of the cone cover on the hub cone with the flared proximal end and the proximal stiffener, according to the present disclosure.

As shown in FIG. 42, the proximal stiffener 520 is adjacent to the flared proximal end 522, which supports the hub cone, with an inner cone 30a therein. A proximal part of the hub cone 30 is surrounded by the cone cover 29.

FIG. 43 is a side view of the cone cover on the hub cone with the flared proximal end and the proximal stiffener, according to the present disclosure.

As shown in FIG. 43, the proximal stiffener 520 is adjacent to the flared proximal end 522, which supports the hub cone. A proximal part of the hub cone 30 is surrounded by the cone cover 29 and support sleeve guide channels 514 extend from a proximal end of the hub cone 30.

Accordingly, an aspect of the present disclosure provides a hub 30 for a continuum robot. The hub may include a distal end 410; a proximal end 430; a section 420 extending from the distal end 410 to the proximal end 430; and a plurality of guide channels 31a, 31b, . . . 31e. Each guide channel of the plurality of guide channels 31a, 31b, . . . 31e may extend along an exterior surface 424 of the section 420. The exterior surface 424 may include a distal pitch diameter change and a proximal pitch diameter change. The distal pitch diameter change may extend from the distal end 410 along a first length 414 of the hub 30. The proximal pitch diameter change may extend from a proximal end of the distal pitch diameter change along a second length 416 of the hub 30.

The hub 30 may be substantially conical in shape, the distal end 410 may have a first face 412 with a first maximum length Lₘₐₓ1, the proximal end 430 may have a second face 432 with a second maximum length Lₘₐₓ2, larger than the first maximum length Lₘₐₓ1, and the first length 414 may be longer than the second length 416.

The distal pitch diameter change may be a substantially concave curve, and the proximal pitch diameter change may be a substantially convex curve.

Another aspect of the present disclosure provides a hub 30 for connecting a continuum robot to a controller, with the hub including a distal end 410; a proximal end 430; and a section 420 between the proximal end 430 and the distal end 410. The distal end 410 may include a first face 412 with a first maximum length Lₘₐₓ1. The proximal end 430 may include a second face 432 with a second maximum length Lₘₐₓ2, larger than the first maximum length Lₘₐₓ1.

The first face 412 may be substantially round or substantially elliptical, with the first maximum length Lₘₐₓ1 being a first diameter. The second face 432 may be substantially round or substantially elliptical. The second maximum length Lₘₐₓ2 may be a second diameter.

The first face 412 may be substantially quadrilateral, with the first maximum length Lₘₐₓ1 being a longest distance between sides of the substantially quadrilateral shape along the first face 412. The second face 432 may be substantially quadrilateral, with the second longest length Lₘₐₓ2 being a longest distance between sides of the substantially quadrilateral shape along the second face 432.

The hub 30 may include a plurality of hub guide channels 31a, 31b, . . . 31e extending along at least a part of a length of an exterior surface 424 of the section 420. Each hub guide channel of the plurality of hub guide channels 31a, 31b may be configured to receive a respective hub hypotube 8 therein.

The hub 30 may include a plurality of depressions 460a, 460b, 460c. The hub hypotube 8 may be received in the respective hub guide channel 31a. An outer surface of the hypotube 8 may be one of flush with or recessed in the exterior surface 424 of the hub 30. With the hub hypotube 8 received in the respective hub guide channel, a portion of an outer surface of the hub hypotube 8 may protrude from the exterior surface 424 of the hub 30.

The hub 30 may include a plurality of depressions 460a, 460b, 460c and a plurality of plateaus 450a, 450b. Each depression of the plurality of depressions 460a, 460b, 460c and each plateau of the plurality of plateaus 450a, 450b may extend along the length of the exterior surface 424. Each hub guide channel of the plurality hub guide channels 31a, 31b, . . . 31e may be positioned between a respective pair of plateaus 450a, 450b. Each depression of the plurality of depressions 460a, 460b, 460c may be positioned between a respective pair of hub guide channels.

The hub 30 may include a plurality of hub hypotubes 8 and a hub cover 29. The plurality of hub hypotubes 8 may extend along a length of an exterior surface 424 of the section 420. The hub cover 29 may be configured to enclose at least part of the exterior surface 424 of the hub 30 with the plurality of hub hypotubes 8 therebetween.

The hub cover 29 may be configured to lock onto the exterior surface 424. The locking prevents longitudinal movement or twisting between the hub cover 29 and the hub 30.

The hub cover 29 may be formed of an elastomer having a durometer scale lower than a durometer scale of the plurality of hub hypotubes 8. The durometer scale of the hub cover 29 may be lower than a durometer scale of the hub 30. When enclosing the at least part of the exterior surface of the hub 30, the hub cover 29 may press the plurality of hub hypotubes 8 against the hub 30 to seal the plurality of hub hypotubes 8 between the hub 30 and the hub cover 29.

The hub 30 may include a shell 34 that includes at least two parts and a tool channel 20. Assembly of the at least two parts of the shell 34 may press an inner surface of the shell 34 against at least a part of an outer surface of the hub cover 29, support an exit port of the tool channel 20, and enclose the plurality of hub hypotubes 8 between the hub 30 and the hub cover 29.

The hub 30 may include a plurality of hub guide channels 31a, 31b, . . . 31e extending along a length of the exterior surface 424 of the section 420. Each hub guide channel of the plurality of hub guide channels 31a, 31b, . . . 31e may be configured to accommodate at least a portion of a respective hub hypotube 8 therein. Each hub guide channel is substantially round in shape, having a diameter equal to or larger than a diameter of the hub hypotube 8 accommodated therein.

The hub may include a hollow 436 extending through the section 420 from the first face 412 to the second face 432.

Another aspect of the present disclosure provides an access component for a continuum robot that may include a shaft 5 including at least nine driving wires 4, a proximal section, and a distal section. The access component may be a housing, a conduit, or other component for removably affixing the continuum robot to a controller. The access component may include a hub 30 configured to fixedly attach to the shaft 5, with the hub being configured to removably attach the continuum robot 100 to a controller; and a hub cover 29. Each driving wire of the at least nine driving wires 4 may extend through a respective hub hypotube 8 from a proximal end 430 of the hub 30. At least one of a posture or a pose of the distal section may change in response to at least one of a push force and a pull force on a proximal end of one or more of the at least nine driving wires 4. A distal end 410 of the hub 30 may include a first face 412 with a diameter having a first maximum length Lₘₐₓ1. The proximal end 430 may include a second face 432 with a diameter having a second maximum length Lₘₐₓ2, larger than the first maximum length Lₘₐₓ1. The hub hypotubes 8 may extend from the proximal end 430 to the distal end 410, across an exterior of the hub. The hub cover 29 may enclose at least part of an exterior surface 424 of the hub 30 with the plurality of hub hypotubes 8 therebetween.

A pitch diameter transition may occur along the exterior surface 424 from the proximal end 430 to the distal end 410. At the proximal end 430, the proximal pitch diameter may substantially align with a diameter of actuator clamps 7 of the controller 2. At the distal end 410, the distal pitch diameter may substantially align with a diameter of the at least nine driving wires 4 in the shaft 5.

The continuum robot may include a stiffener 520 extending from the distal end 410 along a portion of the shaft 5 and a transition T provided within the stiffener adjacent to the distal end 410 of the hub 30. The stiffener 520 may include a flared end 522 configured to extend over a portion of the hub 30, with the flared end referring to an internal diameter of the stiffener 520. The transition T may be configured to attach the hub hypotube to the shaft, with the hub hypotubes being configured to support the at least one driving wire 4 that extends through the hub.

Another aspect of the present disclosure provides a method for manufacturing a continuum robot that includes forming a hub body by aligning a hub cone 30 with a tool channel 20 extending through the hub body and a plurality of hub hypotubes 8 extending along an exterior of the hub body; extending first ends of mandrels of a plurality of mandrels into respective hollows of hub hypotubes 8 of the plurality of hub hypotubes 8; extending second ends of the mandrels into hollows of a respective support sleeve lumen of a plurality of support sleeve lumens 516 that align with the respective hollows of the hub hypotube 8; thermally bonding the hub body to the plurality of support sleeve lumens 516; and forming a channel in each aligned support sleeve lumen and hub hypotube by withdrawing the each mandrel of the plurality of mandrels from the respective hollows. The thermal bonding of the hub body to the plurality of support sleeve lumens 516 may bond the tool channel 20 and the plurality of hub hypotubes to the hub body.

An aspect of the present disclosure provides a hub body formed by aligning a hub cone 30 with a tool channel 20 extending through the hub body and a plurality of hub hypotubes 8 extending along an exterior of the hub body; extending first ends of mandrels of a plurality of mandrels into respective hollows of hub hypotubes 8 of the plurality of hub hypotubes 8; extending second ends of the mandrels into hollows of a respective support sleeve lumen of a plurality of support sleeve lumens 516 that align with the respective hollows of the hub hypotube 8; thermally bonding the hub body to the plurality of support sleeve lumens 516; and forming a channel in each aligned support sleeve lumen and hub hypotube by withdrawing the each mandrel of the plurality of mandrels from the respective hollows. The thermal bonding of the hub body to the plurality of support sleeve lumens 516 may bond the tool channel 20 and the plurality of hub hypotubes to the hub body.

A cone cover may hold the plurality of hub hypotubes 8 in place during the thermal bonding. The plurality of support sleeve lumens 516 may extend into a catheter shaft 5, with a proximal end of the catheter shaft 5 being positioned adjacent to a distal end of the hub cone 30.

The tool channel 20 may extend through a center of the hub cone 30 and a center of the catheter shaft 5. A mandrel of the plurality of mandrels may maintain at least one opening in the tool channel 20 between the hub cone 30 and the catheter shaft 5.

A first portion of the catheter shaft 5 may surround the plurality of support sleeve lumens 516 and a second portion of the catheter shaft may surround a plurality of drive wire lumens 518, with the second portion being distal of the first portion. In the reflow, PTFE coated mandrels may extend through a respective drive wire lumen 518 to support each of the plurality of drive wire lumens.

The thermal bonding may be reflow soldering performed at approximately 180 degrees C. and the plurality of mandrels may be coated with PTFE.

### REFERENCE NUMBERS

| | |
|---|---|
| Actuator | 2 |
| Push/pull drive wires | 4, 111b, 112b, 113b |
| Catheter shaft | 5 |
| Proximal catheter shaft extrusion | 5a |
| Distal catheter shaft extrusion | 5b |
| Hub body | 6 |
| Proximal Hub body | 6a |
| Distal Hub body | 6b |
| Actuator clamp | 7 |
| Hub hypotube | 8 |
| Pusher rod | 9 |
| Pusher hypotube | 10 |
| Hub guide hypotube | 13 |
| Resilient member | 15 |
| Hub guide disks | 19 |
| Tool channel | 20 |
| Hub extrusion | 21 |
| Clamp rod | 23 |
| Hypotube clamp sleeve | 24 |
| Proximal catheter shaft lumen guide | 28 |
| Cone cover | 29 |
| Hub cone | 30 |
| Inner cone | 30a |
| Hub guide channels | 31a, . . . 31h |
| Straight hub channel | 32 |
| Support spring for hub hypotube | 33 |
| Outer shell | 34 |
| Medical device system | 40 |
| Positioning cart | 44 |
| Navigation software | 46 |
| Operation console | 50 |
| Base stage | 52 |
| Continuum robot | 100 |
| Distal bending section | 102 |
| Middle bending section | 104 |
| Proximal bending section | 106 |
| Connection portions | 121, 122, 123 |
| Pitch diameter transition | 400 |
| Distal end of hub cone | 410 |
| First section | 414 |
| Face of the distal end | 412 |
| Second section | 416 |
| Hub cone section | 420 |
| Exterior surface of conical section | 424 |
| Proximal end of hub cone | 430 |
| Face of the proximal end | 432 |
| Hollow through substantially | 436 |
| conical section | |
| Plateaus | 450a, 450b |
| Depressions | 460a, 460b |
| Guide channels hub body | 510 |
| Guide channels hub cone | 514 |
| Support sleeve lumens | 516 |
| Drive wire lumens | 518 |
| Stiffener | 520 |
| Flared proximal end | 522 |
| Tapered distal end | 530 |
| Engaging tab | L_{1B} |
| Receiving tab | L_{1A} |

In referring to the description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. Skilled artisans may employ such variations as appropriate, and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A hub for a continuum robot, the hub comprising:
a distal end;
a proximal end;
a section extending from the distal end to the proximal end; and
a plurality of guide channels, wherein:
each guide channel of the plurality of guide channels extends along a surface of the section,
the surface includes a distal pitch diameter change and a proximal pitch diameter change,
the distal pitch diameter change extends from the distal end along a first length of the hub, and
the proximal pitch diameter change extends from a proximal end of the distal pitch diameter change along a second length of the hub.

2. The hub of claim 1, wherein:
the hub is substantially conical in shape,
the distal end has a first face with a first maximum length, and
the proximal end has a second face with a second maximum length, larger than the first maximum length.

3. The hub of claim 1, wherein:
the distal pitch diameter change is substantially concave, and
the proximal pitch diameter change is substantially convex.

4. A hub for connecting a continuum robot to a controller, the hub comprising:
a distal end;
a proximal end; and
a section between the proximal end and the distal end, wherein:
the distal end includes a first face with a first maximum length, and
the proximal end includes a second face with a second maximum length, larger than the first maximum length.

5. The hub of claim 4, wherein:
the first face is substantially round or substantially elliptical,
the first maximum length is a first diameter,
the second face is substantially round or substantially elliptical, and
the second maximum length is a second diameter.

6. The hub of claim 4, wherein:
the first face is substantially quadrilateral,
the first maximum length is a longest distance between sides of the substantially quadrilateral shape along the first face,
the second face is substantially quadrilateral, and
the second longest length is a longest distance between sides of the substantially quadrilateral shape along the second face.

7. The hub of claim 4, further comprising a plurality of hub guide channels extending along at least a part of a length of a surface of the section.

8. The hub of claim 7, wherein each hub guide channel of the plurality of hub guide channels is configured to receive a respective hub hypotube therein.

9. The hub of claim 8, further comprising:
a plurality of depressions,
wherein, with the hub hypotube received in the respective hub guide channel, an outer surface of the hypotube is one of flush with or recessed in the surface of the hub.

10. The hub of claim 8, wherein, with the hub hypotube received in the respective hub guide channel, a portion of an outer surface of the hub hypotube protrudes from the surface of the hub.

11. The hub of claim 7, further comprising:
a plurality of depressions; and
a plurality of plateaus, wherein:
each depression of the plurality of depressions and each plateau of the plurality of plateaus extends along the length of the surface,
each hub guide channel of the plurality hub guide channels is positioned between a respective pair of plateaus, and
each depression of the plurality of depressions is positioned between a respective pair of hub guide channels.

12. The hub 30 of claim 4, further comprising:
a plurality of hub hypotubes; and
a hub cover, wherein:
the plurality of hub hypotubes extend along a length of a surface of the section, and
the hub cover is configured to enclose at least part of the surface of the hub with the plurality of hub hypotubes therebetween.

13. The hub of claim 12, wherein:
the hub cover is formed of an elastomer having a durometer scale lower than a durometer scale of the plurality of hub hypotubes,
the durometer scale of the hub cover is lower than a durometer scale of the hub, and
when enclosing the at least part of the surface, the hub cover presses the plurality of hub hypotubes against the hub to seal the plurality of hub hypotubes between the hub and the hub cover.

14. The hub of claim 12, further comprising:
a shell that includes at least two parts.

15. The hub 30 of claim 14, further comprising:
a tool channel, wherein assembly of the at least two parts of the shell:
presses an inner surface of the shell against at least a part of an outer surface of the hub cover,
supports an exit port of the tool channel, and
encloses the plurality of hub hypotubes between the hub and the hub cover.

16. The hub of claim 14, wherein:
the at least two parts of the shell are configured to lock together and surround the surface, and
the locking prevents longitudinal movement or twisting of the hub cover and the hub.

17. The hub of claim 12, further comprising:
a shell, wherein:
the hub cover is formed of an elastomer having a durometer scale lower than a durometer scale of the plurality of hub hypotubes and lower than a durometer scale of the hub,
the shell is formed of an elastomer having a durometer scale higher than the durometer scale of the hub cover, and
when enclosing the at least part of the surface:
the shell presses the hub cover against the hub, and
the hub cover presses the plurality of hub hypotubes against the hub.

18. The hub of claim 17, wherein the pressing of the plurality of hub hypotubes against the hub seals the plurality of hub hypotubes between the hub and the hub cover.

19. The hub of claim 12, further comprising:
a plurality of hub guide channels extending along a length of the surface of the section, wherein
each hub guide channel of the plurality of hub guide channels is configured to accommodate at least a portion of a respective hub hypotube therein.

20. The hub of claim 19, wherein each hub guide channel has a diameter substantially equal to or larger than a diameter of the hub hypotube accommodated therein.

21. The hub of claim 4, further comprising a hollow extending through the section from the first face to the second face.

22. An access component for a continuum robot, the access component comprising:
a hub configured to fixedly attach to a shaft of the continuum robot, wherein the hub is configured to attach the continuum robot to a controller; and
a hub cover, wherein:
a distal end of the hub includes a first face with a diameter having a first maximum length,
a proximal end of the hub includes a second face with a diameter having a second maximum length, larger than the first maximum length, and
hub hypotubes extending from the proximal end to the distal end, across the hub.

23. The access component of claim 22, wherein:
a pitch diameter transition occurs along a surface from the proximal end to the distal end,
at the proximal end, the proximal pitch diameter substantially aligns with a diameter of actuator clamps of the controller, and
at the distal end, the distal pitch diameter substantially aligns with a diameter of at least two driving wires extending through the shaft.

24. The access component of claim 22, further comprising:
a stiffener extending from the distal end along a portion of the shaft; and
a transition provided within the stiffener adjacent to the distal end of the hub, wherein:
the stiffener includes a flared end configured to extend over a portion of the hub, and
the transition is configured to attach the hub hypotube to the shaft, with the hub hypotube being configured to support at least one driving wire that extends through the hub.

25. The access component of claim 22, wherein:
the hub cover encloses at least a proximal part of a surface of the hub with the plurality of hub hypotubes therebetween.

26. The access component of claim 22, wherein:
the shaft is configured to accommodate at least two driving wires therein,
each driving wire of the at least two driving wires extends through a respective hub hypotube from a proximal end of the hub, and
at least one of a posture or a pose of the continuum robot changes in response to at least one of a push force and a pull force on a proximal end of one or more of the at least two driving wires.

27. The access component of claim 22, further comprising:
a shell, wherein:
the hub cover is formed of an elastomer having a durometer scale lower than a durometer scale of the hub hypotubes and lower than a durometer scale of the hub,
the shell is formed of an elastomer having a durometer scale higher than the durometer scale of the hub cover, and
when enclosing at least part of the hub, the shell presses the hub cover against the hub and the hub cover presses the plurality of hub hypotubes against the hub.
